SEQUENCE LISTING (333 sequences)

SEQ ID NO: 1 - DNA, 12 bp, Artificial Sequence
  Description of Artificial Sequence Synthetic oligonucleotide motif sequence
acgattaaaaga

SEQ ID NO: 2 - DNA, 12 bp, Artificial Sequence
  Description of Artificial Sequence Synthetic oligonucleotide motif sequence
tcttttaatcgt

SEQ ID NO: 3 - DNA, 13 bp, Artificial Sequence
  Description of Artificial Sequence Synthetic oligonucleotide motif sequence
aacgattaaaaga

SEQ ID NO: 4 - DNA, 13 bp, Artificial Sequence
  Description of Artificial Sequence Synthetic oligonucleotide motif sequence
tcttttaatcgtt

SEQ ID NO: 5 - DNA, 12 bp, Artificial Sequence
  Description of Artificial Sequence Synthetic oligonucleotide motif sequence
agaaaaggtgac

SEQ ID NO: 6 - DNA, 12 bp, Artificial Sequence
  Description of Artificial Sequence Synthetic oligonucleotide motif sequence
gtcaccttttct

SEQ ID NO: 7 - DNA, 12 bp, Artificial Sequence
  Description of Artificial Sequence Synthetic oligonucleotide motif sequence
acgtcaattatc

SEQ ID NO: 8 - DNA, 12 bp, Artificial Sequence
  Description of Artificial Sequence Synthetic oligonucleotide motif sequence
atactcatgaac

SEQ ID NO: 9 - DNA, 12 bp, Artificial Sequence
  Description of Artificial Sequence Synthetic oligonucleotide motif sequence
gataattgacgt

SEQ ID NO: 10 - DNA, 12 bp, Artificial Sequence
  Description of Artificial Sequence Synthetic oligonucleotide motif sequence
gttcatgagtat

SEQ ID NO: 11 - DNA, 13 bp, Artificial Sequence
  Description of Artificial Sequence Synthetic oligonucleotide motif sequence
aaacgattaaaag

SEQ ID NO: 12 - DNA, 13 bp, Artificial Sequence
  Description of Artificial Sequence Synthetic oligonucleotide motif sequence
atgcaagcctacc

SEQ ID NO: 13 - DNA, 13 bp, Artificial Sequence
  Description of Artificial Sequence Synthetic oligonucleotide motif sequence
cttttaatcgttt

SEQ ID NO: 14 - DNA, 13 bp, Artificial Sequence
  Description of Artificial Sequence Synthetic oligonucleotide motif sequence
ggtaggcttgcat

SEQ ID NO: 15 - DNA, 13 bp, Artificial Sequence
  Description of Artificial Sequence Synthetic oligonucleotide motif sequence
taacttaaaataa

SEQ ID NO: 16 - DNA, 13 bp, Artificial Sequence
  Description of Artificial Sequence Synthetic oligonucleotide motif sequence
tacttactgctaa

SEQ ID NO: 17 - DNA, 13 bp, Artificial Sequence
  Description of Artificial Sequence Synthetic oligonucleotide motif sequence
ttagcagtaagta

SEQ ID NO: 18 - DNA, 13 bp, Artificial Sequence
  Description of Artificial Sequence Synthetic oligonucleotide motif sequence
ttattttaagtta

SEQ ID NO: 19 - DNA, 14 bp, Artificial Sequence
  Description of Artificial Sequence Synthetic oligonucleotide motif sequence
aaacgattaaaaga

SEQ ID NO: 20 - DNA, 14 bp, Artificial Sequence
  Description of Artificial Sequence Synthetic oligonucleotide motif sequence
aaagaaatgattgt

SEQ ID NO: 21 - DNA, 14 bp, Artificial Sequence
  Description of Artificial Sequence Synthetic oligonucleotide motif sequence
acaatcatttcttt

SEQ ID NO: 22 - DNA, 14 bp, Artificial Sequence
  Description of Artificial Sequence Synthetic oligonucleotide motif sequence
aggtaggcttgcat

SEQ ID NO: 23 - DNA, 14 bp, Artificial Sequence
  Description of Artificial Sequence Synthetic oligonucleotide motif sequence
atgcaagcctacct

SEQ ID NO: 24 - DNA, 14 bp, Artificial Sequence
  Description of Artificial Sequence Synthetic oligonucleotide motif sequence
gttattttaagtta

SEQ ID NO: 25 - DNA, 14 bp, Artificial Sequence
  Description of Artificial Sequence Synthetic oligonucleotide motif sequence
taacttaaaataac

SEQ ID NO: 26 - DNA, 14 bp, Artificial Sequence
  Description of Artificial Sequence Synthetic oligonucleotide motif sequence
tcttttaatcgttt

SEQ ID NO: 27 - DNA, 12 bp, Artificial Sequence
  Description of Artificial Sequence Synthetic oligonucleotide motif sequence
aaataagggagt

SEQ ID NO: 28 - DNA, 12 bp, Artificial Sequence
  Description of Artificial Sequence Synthetic oligonucleotide motif sequence
actcccttattt

SEQ ID NO: 29 - DNA, 12 bp, Artificial Sequence
  Description of Artificial Sequence Synthetic oligonucleotide motif sequence
gtaatttactta

SEQ ID NO: 30 - DNA, 12 bp, Artificial Sequence
  Description of Artificial Sequence Synthetic oligonucleotide motif sequence
taagcagtaata

SEQ ID NO: 31 - DNA, 12 bp, Artificial Sequence
  Description of Artificial Sequence Synthetic oligonucleotide motif sequence
taagtaaattac

SEQ ID NO: 32 - DNA, 12 bp, Artificial Sequence
  Description of Artificial Sequence Synthetic oligonucleotide motif sequence
tattactgctta

SEQ ID NO: 33 - DNA, 11 bp, Artificial Sequence
  Description of Artificial Sequence Synthetic oligonucleotide motif sequence
tactctataca

SEQ ID NO: 34 - DNA, 11 bp, Artificial Sequence
  Description of Artificial Sequence Synthetic oligonucleotide motif sequence
tgtatagagta

SEQ ID NO: 35 - DNA, 12 bp, Artificial Sequence
  Description of Artificial Sequence Synthetic oligonucleotide motif sequence
agagcttattca

SEQ ID NO: 36 - DNA, 12 bp, Artificial Sequence
  Description of Artificial Sequence Synthetic oligonucleotide motif sequence
tgaataagctct

SEQ ID NO: 37 - DNA, 13 bp, Artificial Sequence
  Description of Artificial Sequence Synthetic oligonucleotide motif sequence
agagcttattcaa

SEQ ID NO: 38 - DNA, 13 bp, Artificial Sequence
  Description of Artificial Sequence Synthetic oligonucleotide motif sequence
ctagaaaaaacag

SEQ ID NO: 39 - DNA, 13 bp, Artificial Sequence
  Description of Artificial Sequence Synthetic oligonucleotide motif sequence
ctgttttttctag

SEQ ID NO: 40 - DNA, 13 bp, Artificial Sequence
  Description of Artificial Sequence Synthetic oligonucleotide motif sequence
ttgaataagctct

SEQ ID NO: 41 - DNA, 12 bp, Artificial Sequence
  Description of Artificial Sequence Synthetic oligonucleotide motif sequence
aaaaataaggga

SEQ ID NO: 42 - DNA, 12 bp, Artificial Sequence
  Description of Artificial Sequence Synthetic oligonucleotide motif sequence
tcccttattttt

SEQ ID NO: 43 - DNA, 12 bp, Artificial Sequence
  Description of Artificial Sequence Synthetic oligonucleotide motif sequence
cccttattttta

SEQ ID NO: 44 - DNA, 12 bp, Artificial Sequence
  Description of Artificial Sequence Synthetic oligonucleotide motif sequence
taaaaataaggg

SEQ ID NO: 45 - DNA, 11 bp, Artificial Sequence
  Description of Artificial Sequence Synthetic oligonucleotide motif sequence
cgatagagtca

SEQ ID NO: 46 - DNA, 11 bp, Artificial Sequence
  Description of Artificial Sequence Synthetic oligonucleotide motif sequence
tgactctatcg

SEQ ID NO: 47 - DNA, 12 bp, Artificial Sequence
  Description of Artificial Sequence Synthetic oligonucleotide motif sequence
ccgatagagtca

SEQ ID NO: 48 - DNA, 12 bp, Artificial Sequence
  Description of Artificial Sequence Synthetic oligonucleotide motif sequence
tgactctatcgg

SEQ ID NO: 49 - DNA, 12 bp, Artificial Sequence
  Description of Artificial Sequence Synthetic oligonucleotide motif sequence
gtagctgctgct

SEQ ID NO: 50 - DNA, 12 bp, Artificial Sequence
  Description of Artificial Sequence Synthetic oligonucleotide motif sequence
agcagcagctac

SEQ ID NO: 51 - DNA, 12 bp, Artificial Sequence
  Description of Artificial Sequence Synthetic oligonucleotide motif sequence
cgatagagtcac

SEQ ID NO: 52 - DNA, 12 bp, Artificial Sequence
  Description of Artificial Sequence Synthetic oligonucleotide motif sequence
gtgactctatcg

SEQ ID NO: 53 - DNA, 12 bp, Artificial Sequence
  Description of Artificial Sequence Synthetic oligonucleotide motif sequence
aaacccatcaag

SEQ ID NO: 54 - DNA, 12 bp, Artificial Sequence
  Description of Artificial Sequence Synthetic oligonucleotide motif sequence
cttgatgggttt

SEQ ID NO: 55 - DNA, 13 bp, Artificial Sequence
  Description of Artificial Sequence Synthetic oligonucleotide motif sequence
gtagctgctgctg

SEQ ID NO: 56 - DNA, 13 bp, Artificial Sequence
  Description of Artificial Sequence Synthetic oligonucleotide motif sequence
cagcagcagctac

SEQ ID NO: 57 - DNA, 13 bp, Artificial Sequence
  Description of Artificial Sequence Synthetic oligonucleotide motif sequence
gtgactctatcgg

SEQ ID NO: 58 - DNA, 13 bp, Artificial Sequence
  Description of Artificial Sequence Synthetic oligonucleotide motif sequence
ccgatagagtcac

SEQ ID NO: 59 - DNA, 11 bp, Artificial Sequence
  Description of Artificial Sequence Synthetic oligonucleotide motif sequence
cacatcaaagg

SEQ ID NO: 60 - DNA, 11 bp, Artificial Sequence
  Description of Artificial Sequence Synthetic oligonucleotide motif sequence
ctcagtcctga

SEQ ID NO: 61 - DNA, 11 bp, Artificial Sequence
  Description of Artificial Sequence Synthetic oligonucleotide motif sequence
caggactgagt

SEQ ID NO: 62 - DNA, 11 bp, Artificial Sequence
  Description of Artificial Sequence Synthetic oligonucleotide motif sequence
actcagtcctg

SEQ ID NO: 63 - DNA, 11 bp, Artificial Sequence
  Description of Artificial Sequence Synthetic oligonucleotide motif sequence
aggtaatttcc

SEQ ID NO: 64 - DNA, 11 bp, Artificial Sequence
  Description of Artificial Sequence Synthetic oligonucleotide motif sequence
tcaggactgag

SEQ ID NO: 65 - DNA, 11 bp, Artificial Sequence
  Description of Artificial Sequence Synthetic oligonucleotide motif sequence
cctttgatgtg

SEQ ID NO: 66 - DNA, 11 bp, Artificial Sequence
  Description of Artificial Sequence Synthetic oligonucleotide motif sequence
ggaaattacct

SEQ ID NO: 67 - DNA, 12 bp, Artificial Sequence
  Description of Artificial Sequence Synthetic oligonucleotide motif sequence
gtttggctacca

SEQ ID NO: 68 - DNA, 12 bp, Artificial Sequence
  Description of Artificial Sequence Synthetic oligonucleotide motif sequence
tcaggactgagt

SEQ ID NO: 69 - DNA, 12 bp, Artificial Sequence
  Description of Artificial Sequence Synthetic oligonucleotide motif sequence
caggactgagtt

SEQ ID NO: 70 - DNA, 12 bp, Artificial Sequence
  Description of Artificial Sequence Synthetic oligonucleotide motif sequence
aggtaatttccc

SEQ ID NO: 71 - DNA, 12 bp, Artificial Sequence
  Description of Artificial Sequence Synthetic oligonucleotide motif sequence
gtccttgctaaa

SEQ ID NO: 72 - DNA, 12 bp, Artificial Sequence
  Description of Artificial Sequence Synthetic oligonucleotide motif sequence
ttcacgggcaat

SEQ ID NO: 73 - DNA, 12 bp, Artificial Sequence
  Description of Artificial Sequence Synthetic oligonucleotide motif sequence
actcagtcctga

SEQ ID NO: 74 - DNA, 12 bp, Artificial Sequence
  Description of Artificial Sequence Synthetic oligonucleotide motif sequence
attgcccgtgaa

SEQ ID NO: 75 - DNA, 12 bp, Artificial Sequence
  Description of Artificial Sequence Synthetic oligonucleotide motif sequence
aactcagtcctg

SEQ ID NO: 76 - DNA, 12 bp, Artificial Sequence
  Description of Artificial Sequence Synthetic oligonucleotide motif sequence
gggaaattacct

SEQ ID NO: 77 - DNA, 12 bp, Artificial Sequence
  Description of Artificial Sequence Synthetic oligonucleotide motif sequence
tggtagccaaac

SEQ ID NO: 78 - DNA, 12 bp, Artificial Sequence
  Description of Artificial Sequence Synthetic oligonucleotide motif sequence
tttagcaaggac

SEQ ID NO: 79 - DNA, 12 bp, Artificial Sequence
  Description of Artificial Sequence Synthetic oligonucleotide motif sequence
accggagctttc

SEQ ID NO: 80 - DNA, 12 bp, Artificial Sequence
  Description of Artificial Sequence Synthetic oligonucleotide motif sequence
gaaagctccggt

SEQ ID NO: 81 - DNA, 13 bp, Artificial Sequence
  Description of Artificial Sequence Synthetic oligonucleotide motif sequence
atgaagtatttct

SEQ ID NO: 82 - DNA, 13 bp, Artificial Sequence
  Description of Artificial Sequence Synthetic oligonucleotide motif sequence
aactcagtcctga

SEQ ID NO: 83 - DNA, 13 bp, Artificial Sequence
  Description of Artificial Sequence Synthetic oligonucleotide motif sequence
catctgatacacc

SEQ ID NO: 84 - DNA, 13 bp, Artificial Sequence
  Description of Artificial Sequence Synthetic oligonucleotide motif sequence
ggtgtatcagatg

SEQ ID NO: 85 - DNA, 13 bp, Artificial Sequence
  Description of Artificial Sequence Synthetic oligonucleotide motif sequence
agaaatacttcat

SEQ ID NO: 86 - DNA, 13 bp, Artificial Sequence
  Description of Artificial Sequence Synthetic oligonucleotide motif sequence
tcaggactgagtt

SEQ ID NO: 87 - DNA, 13 bp, Artificial Sequence
  Description of Artificial Sequence Synthetic oligonucleotide motif sequence
gaaagctccggta

SEQ ID NO: 88 - DNA, 13 bp, Artificial Sequence
  Description of Artificial Sequence Synthetic oligonucleotide motif sequence
taccggagctttc

SEQ ID NO: 89 - DNA, 14 bp, Artificial Sequence
  Description of Artificial Sequence Synthetic oligonucleotide motif sequence
agatatggaagaag

SEQ ID NO: 90 - DNA, 14 bp, Artificial Sequence
  Description of Artificial Sequence Synthetic oligonucleotide motif sequence
cttcttccatatct

SEQ ID NO: 91 - DNA, 11 bp, Artificial Sequence
  Description of Artificial Sequence Synthetic oligonucleotide motif sequence
tctggttagca

SEQ ID NO: 92 - DNA, 11 bp, Artificial Sequence
  Description of Artificial Sequence Synthetic oligonucleotide motif sequence
atgcagagagt

SEQ ID NO: 93 - DNA, 11 bp, Artificial Sequence
  Description of Artificial Sequence Synthetic oligonucleotide motif sequence
ccgtgtaatgg

SEQ ID NO: 94 - DNA, 11 bp, Artificial Sequence
  Description of Artificial Sequence Synthetic oligonucleotide motif sequence
tgctaaccaga

SEQ ID NO: 95 - DNA, 11 bp, Artificial Sequence
  Description of Artificial Sequence Synthetic oligonucleotide motif sequence
ggttatacaga

SEQ ID NO: 96 - DNA, 11 bp, Artificial Sequence
  Description of Artificial Sequence Synthetic oligonucleotide motif sequence
tctgtataacc

SEQ ID NO: 97 - DNA, 11 bp, Artificial Sequence
  Description of Artificial Sequence Synthetic oligonucleotide motif sequence
actctctgcat

SEQ ID NO: 98 - DNA, 11 bp, Artificial Sequence
  Description of Artificial Sequence Synthetic oligonucleotide motif sequence
ttagctcgcat

SEQ ID NO: 99 - DNA, 11 bp, Artificial Sequence
  Description of Artificial Sequence Synthetic oligonucleotide motif sequence
atgcgagctaa

SEQ ID NO: 100 - DNA, 11 bp, Artificial Sequence
  Description of Artificial Sequence Synthetic oligonucleotide motif sequence
ccattacacgg

SEQ ID NO: 101 - DNA, 12 bp, Artificial Sequence
  Description of Artificial Sequence Synthetic oligonucleotide motif sequence
gcattaagcatg

SEQ ID NO: 102 - DNA, 12 bp, Artificial Sequence
  Description of Artificial Sequence Synthetic oligonucleotide motif sequence
ccattacacggc

SEQ ID NO: 103 - DNA, 12 bp, Artificial Sequence
  Description of Artificial Sequence Synthetic oligonucleotide motif sequence
tcaagacccagg

SEQ ID NO: 104 - DNA, 12 bp, Artificial Sequence
  Description of Artificial Sequence Synthetic oligonucleotide motif sequence
gccgtgtaatgg

SEQ ID NO: 105 - DNA, 12 bp, Artificial Sequence
  Description of Artificial Sequence Synthetic oligonucleotide motif sequence
gagatatctttt

SEQ ID NO: 106 - DNA, 12 bp, Artificial Sequence
  Description of Artificial Sequence Synthetic oligonucleotide motif sequence
aaaagatatctc

SEQ ID NO: 107 - DNA, 12 bp, Artificial Sequence
  Description of Artificial Sequence Synthetic oligonucleotide motif sequence
cctgggtcttga

SEQ ID NO: 108 - DNA, 12 bp, Artificial Sequence
  Description of Artificial Sequence Synthetic oligonucleotide motif sequence
cacatcaaaggc

SEQ ID NO: 109 - DNA, 12 bp, Artificial Sequence
  Description of Artificial Sequence Synthetic oligonucleotide motif sequence
gagcaggacttt

SEQ ID NO: 110 - DNA, 12 bp, Artificial Sequence
  Description of Artificial Sequence Synthetic oligonucleotide motif sequence
gctctttgcttc

SEQ ID NO: 111 - DNA, 12 bp, Artificial Sequence
  Description of Artificial Sequence Synthetic oligonucleotide motif sequence
acttacaccaaa

SEQ ID NO: 112 - DNA, 12 bp, Artificial Sequence
  Description of Artificial Sequence Synthetic oligonucleotide motif sequence
gcctttgatgtg

SEQ ID NO: 113 - DNA, 12 bp, Artificial Sequence
  Description of Artificial Sequence Synthetic oligonucleotide motif sequence
gaagcaaagagc

SEQ ID NO: 114 - DNA, 12 bp, Artificial Sequence
  Description of Artificial Sequence Synthetic oligonucleotide motif sequence
tttggtgtaagt

SEQ ID NO: 115 - DNA, 12 bp, Artificial Sequence
  Description of Artificial Sequence Synthetic oligonucleotide motif sequence
atactcaccaaa

SEQ ID NO: 116 - DNA, 12 bp, Artificial Sequence
  Description of Artificial Sequence Synthetic oligonucleotide motif sequence
catgcttaatgc

SEQ ID NO: 117 - DNA, 12 bp, Artificial Sequence
  Description of Artificial Sequence Synthetic oligonucleotide motif sequence
aaagtcctgctc

SEQ ID NO: 118 - DNA, 12 bp, Artificial Sequence
  Description of Artificial Sequence Synthetic oligonucleotide motif sequence
tccattgcagaa

SEQ ID NO: 119 - DNA, 12 bp, Artificial Sequence
  Description of Artificial Sequence Synthetic oligonucleotide motif sequence
tttggtgagtat

SEQ ID NO: 120 - DNA, 12 bp, Artificial Sequence
  Description of Artificial Sequence Synthetic oligonucleotide motif sequence
tcagaagttctc

SEQ ID NO: 121 - DNA, 12 bp, Artificial Sequence
  Description of Artificial Sequence Synthetic oligonucleotide motif sequence
ttctgcaatgga

SEQ ID NO: 122 - DNA, 12 bp, Artificial Sequence
  Description of Artificial Sequence Synthetic oligonucleotide motif sequence
gagaacttctga

SEQ ID NO: 123 - DNA, 13 bp, Artificial Sequence
  Description of Artificial Sequence Synthetic oligonucleotide motif sequence
gatacaggtttat

SEQ ID NO: 124 - DNA, 13 bp, Artificial Sequence
  Description of Artificial Sequence Synthetic oligonucleotide motif sequence
aagcttcttgaaa

SEQ ID NO: 125 - DNA, 13 bp, Artificial Sequence
  Description of Artificial Sequence Synthetic oligonucleotide motif sequence
tcaagacccaggc

SEQ ID NO: 126 - DNA, 13 bp, Artificial Sequence
  Description of Artificial Sequence Synthetic oligonucleotide motif sequence
tgtccttgctaaa

SEQ ID NO: 127 - DNA, 13 bp, Artificial Sequence
  Description of Artificial Sequence Synthetic oligonucleotide motif sequence
agaaaagttgctg

SEQ ID NO: 128 - DNA, 13 bp, Artificial Sequence
  Description of Artificial Sequence Synthetic oligonucleotide motif sequence
gcctgggtcttga

SEQ ID NO: 129 - DNA, 13 bp, Artificial Sequence
  Description of Artificial Sequence Synthetic oligonucleotide motif sequence
agcaggactttgg

SEQ ID NO: 130 - DNA, 13 bp, Artificial Sequence
  Description of Artificial Sequence Synthetic oligonucleotide motif sequence
ataaacctgtatc

SEQ ID NO: 131 - DNA, 13 bp, Artificial Sequence
  Description of Artificial Sequence Synthetic oligonucleotide motif sequence
tcaaacccatcaa

SEQ ID NO: 132 - DNA, 13 bp, Artificial Sequence
  Description of Artificial Sequence Synthetic oligonucleotide motif sequence
tttcaagaagctt

SEQ ID NO: 133 - DNA, 13 bp, Artificial Sequence
  Description of Artificial Sequence Synthetic oligonucleotide motif sequence
caaagtcctgctc

SEQ ID NO: 134 - DNA, 13 bp, Artificial Sequence
  Description of Artificial Sequence Synthetic oligonucleotide motif sequence
agatatttgaagt

SEQ ID NO: 135 - DNA, 13 bp, Artificial Sequence
  Description of Artificial Sequence Synthetic oligonucleotide motif sequence
cagcaacttttct

SEQ ID NO: 136 - DNA, 13 bp, Artificial Sequence
  Description of Artificial Sequence Synthetic oligonucleotide motif sequence
acttcaaatatct

SEQ ID NO: 137 - DNA, 13 bp, Artificial Sequence
  Description of Artificial Sequence Synthetic oligonucleotide motif sequence
gagcaggactttg

SEQ ID NO: 138 - DNA, 13 bp, Artificial Sequence
  Description of Artificial Sequence Synthetic oligonucleotide motif sequence
tttagcaaggaca

SEQ ID NO: 139 - DNA, 13 bp, Artificial Sequence
  Description of Artificial Sequence Synthetic oligonucleotide motif sequence
ccaaagtcctgct

SEQ ID NO: 140 - DNA, 13 bp, Artificial Sequence
  Description of Artificial Sequence Synthetic oligonucleotide motif sequence
gagatatcttttg

SEQ ID NO: 141 - DNA, 13 bp, Artificial Sequence
  Description of Artificial Sequence Synthetic oligonucleotide motif sequence
ttgatgggtttga

SEQ ID NO: 142 - DNA, 13 bp, Artificial Sequence
  Description of Artificial Sequence Synthetic oligonucleotide motif sequence
caaaagatatctc

SEQ ID NO: 143 - DNA, 14 bp, Artificial Sequence
  Description of Artificial Sequence Synthetic oligonucleotide motif sequence
gagcaggactttgg

SEQ ID NO: 144 - DNA, 14 bp, Artificial Sequence
  Description of Artificial Sequence Synthetic oligonucleotide motif sequence
cagcaacttttctt

SEQ ID NO: 145 - DNA, 14 bp, Artificial Sequence
  Description of Artificial Sequence Synthetic oligonucleotide motif sequence
ccaaagtcctgctc

SEQ ID NO: 146 - DNA, 14 bp, Artificial Sequence
  Description of Artificial Sequence Synthetic oligonucleotide motif sequence
aagaaaagttgctg

SEQ ID NO: 147 - DNA, 14 bp, Artificial Sequence
  Description of Artificial Sequence Synthetic oligonucleotide motif sequence
ttaacaaaataact

SEQ ID NO: 148 - DNA, 14 bp, Artificial Sequence
  Description of Artificial Sequence Synthetic oligonucleotide motif sequence
agttattttgttaa

SEQ ID NO: 149 - DNA, 15 bp, Artificial Sequence
  Description of Artificial Sequence Synthetic oligonucleotide motif sequence
tcttcttttatctca

SEQ ID NO: 150 - DNA, 15 bp, Artificial Sequence
  Description of Artificial Sequence Synthetic oligonucleotide motif sequence
tgagataaaagaaga

SEQ ID NO: 151 - DNA, 11 bp, Artificial Sequence
  Description of Artificial Sequence Synthetic oligonucleotide motif sequence
tgcagagtacc

SEQ ID NO: 152 - DNA, 11 bp, Artificial Sequence
  Description of Artificial Sequence Synthetic oligonucleotide motif sequence
attgttcgtgc

SEQ ID NO: 153 - DNA, 11 bp, Artificial Sequence
  Description of Artificial Sequence Synthetic oligonucleotide motif sequence
ctacgctccta

SEQ ID NO: 154 - DNA, 11 bp, Artificial Sequence
  Description of Artificial Sequence Synthetic oligonucleotide motif sequence
taggtactctg

SEQ ID NO: 155 - DNA, 11 bp, Artificial Sequence
  Description of Artificial Sequence Synthetic oligonucleotide motif sequence
taggagcgtag

SEQ ID NO: 156 - DNA, 11 bp, Artificial Sequence
  Description of Artificial Sequence Synthetic oligonucleotide motif sequence
aagtactatgc

SEQ ID NO: 157 - DNA, 11 bp, Artificial Sequence
  Description of Artificial Sequence Synthetic oligonucleotide motif sequence
ggtactctgca

SEQ ID NO: 158 - DNA, 11 bp, Artificial Sequence
  Description of Artificial Sequence Synthetic oligonucleotide motif sequence
cagagtaccta

SEQ ID NO: 159 - DNA, 11 bp, Artificial Sequence
  Description of Artificial Sequence Synthetic oligonucleotide motif sequence
cttactgcgag

SEQ ID NO: 160 - DNA, 11 bp, Artificial Sequence
  Description of Artificial Sequence Synthetic oligonucleotide motif sequence
caccttctcta

SEQ ID NO: 161 - DNA, 11 bp, Artificial Sequence
  Description of Artificial Sequence Synthetic oligonucleotide motif sequence
gcacgaacaat

SEQ ID NO: 162 - DNA, 11 bp, Artificial Sequence
  Description of Artificial Sequence Synthetic oligonucleotide motif sequence
cactaactgat

SEQ ID NO: 163 - DNA, 11 bp, Artificial Sequence
  Description of Artificial Sequence Synthetic oligonucleotide motif sequence
atcagttagtg

SEQ ID NO: 164 - DNA, 11 bp, Artificial Sequence
  Description of Artificial Sequence Synthetic oligonucleotide motif sequence
gtaggagcgta

SEQ ID NO: 165 - DNA, 11 bp, Artificial Sequence
  Description of Artificial Sequence Synthetic oligonucleotide motif sequence
ctcgcagtaag

SEQ ID NO: 166 - DNA, 11 bp, Artificial Sequence
  Description of Artificial Sequence Synthetic oligonucleotide motif sequence
tagagaaggtg

SEQ ID NO: 167 - DNA, 11 bp, Artificial Sequence
  Description of Artificial Sequence Synthetic oligonucleotide motif sequence
tacgctcctac

SEQ ID NO: 168 - DNA, 11 bp, Artificial Sequence
  Description of Artificial Sequence Synthetic oligonucleotide motif sequence
gcatagtactt

SEQ ID NO: 169 - DNA, 12 bp, Artificial Sequence
  Description of Artificial Sequence Synthetic oligonucleotide motif sequence
cttatgacgaac

SEQ ID NO: 170 - DNA, 12 bp, Artificial Sequence
  Description of Artificial Sequence Synthetic oligonucleotide motif sequence
ggctcatctgta

SEQ ID NO: 171 - DNA, 12 bp, Artificial Sequence
  Description of Artificial Sequence Synthetic oligonucleotide motif sequence
gaaccagaaagt

SEQ ID NO: 172 - DNA, 12 bp, Artificial Sequence
  Description of Artificial Sequence Synthetic oligonucleotide motif sequence
actttctggttc

SEQ ID NO: 173 - DNA, 12 bp, Artificial Sequence
  Description of Artificial Sequence Synthetic oligonucleotide motif sequence
tatgtttcagaa

SEQ ID NO: 174 - DNA, 12 bp, Artificial Sequence
  Description of Artificial Sequence Synthetic oligonucleotide motif sequence
gctgggacaagt

SEQ ID NO: 175 - DNA, 12 bp, Artificial Sequence
  Description of Artificial Sequence Synthetic oligonucleotide motif sequence
gcatggctaacc

SEQ ID NO: 176 - DNA, 12 bp, Artificial Sequence
  Description of Artificial Sequence Synthetic oligonucleotide motif sequence
catgcagagagt

SEQ ID NO: 177 - DNA, 12 bp, Artificial Sequence
  Description of Artificial Sequence Synthetic oligonucleotide motif sequence
tgcgtaaccatg

SEQ ID NO: 178 - DNA, 12 bp, Artificial Sequence
  Description of Artificial Sequence Synthetic oligonucleotide motif sequence
ttcagcaagaac

SEQ ID NO: 179 - DNA, 12 bp, Artificial Sequence
  Description of Artificial Sequence Synthetic oligonucleotide motif sequence
aagtctgataac

SEQ ID NO: 180 - DNA, 12 bp, Artificial Sequence
  Description of Artificial Sequence Synthetic oligonucleotide motif sequence
ctgcccagtgat

SEQ ID NO: 181 - DNA, 12 bp, Artificial Sequence
  Description of Artificial Sequence Synthetic oligonucleotide motif sequence
tgggtgctctct

SEQ ID NO: 182 - DNA, 12 bp, Artificial Sequence
  Description of Artificial Sequence Synthetic oligonucleotide motif sequence
gcatagtacttg

SEQ ID NO: 183 - DNA, 12 bp, Artificial Sequence
  Description of Artificial Sequence Synthetic oligonucleotide motif sequence
tctctgcatgta

SEQ ID NO: 184 - DNA, 12 bp, Artificial Sequence
  Description of Artificial Sequence Synthetic oligonucleotide motif sequence
catggttacgca

SEQ ID NO: 185 - DNA, 12 bp, Artificial Sequence
  Description of Artificial Sequence Synthetic oligonucleotide motif sequence
gattttaccatt

SEQ ID NO: 186 - DNA, 12 bp, Artificial Sequence
  Description of Artificial Sequence Synthetic oligonucleotide motif sequence
gttcgtcataag

SEQ ID NO: 187 - DNA, 12 bp, Artificial Sequence
  Description of Artificial Sequence Synthetic oligonucleotide motif sequence
ggttacgcatcg

SEQ ID NO: 188 - DNA, 12 bp, Artificial Sequence
  Description of Artificial Sequence Synthetic oligonucleotide motif sequence
cagatgctttga

SEQ ID NO: 189 - DNA, 12 bp, Artificial Sequence
  Description of Artificial Sequence Synthetic oligonucleotide motif sequence
tgcagagtacct

SEQ ID NO: 190 - DNA, 12 bp, Artificial Sequence
  Description of Artificial Sequence Synthetic oligonucleotide motif sequence
tacagatgagcc

SEQ ID NO: 191 - DNA, 12 bp, Artificial Sequence
  Description of Artificial Sequence Synthetic oligonucleotide motif sequence
aatggtaaaatc

SEQ ID NO: 192 - DNA, 12 bp, Artificial Sequence
  Description of Artificial Sequence Synthetic oligonucleotide motif sequence
ccttatgacgaa

SEQ ID NO: 193 - DNA, 12 bp, Artificial Sequence
  Description of Artificial Sequence Synthetic oligonucleotide motif sequence
gttcttgctgaa

SEQ ID NO: 194 - DNA, 12 bp, Artificial Sequence
  Description of Artificial Sequence Synthetic oligonucleotide motif sequence
caagtactatgc

SEQ ID NO: 195 - DNA, 12 bp, Artificial Sequence
  Description of Artificial Sequence Synthetic oligonucleotide motif sequence
gtaggagcgtag

SEQ ID NO: 196 - DNA, 12 bp, Artificial Sequence
  Description of Artificial Sequence Synthetic oligonucleotide motif sequence
acaaagaccttg

SEQ ID NO: 197 - DNA, 12 bp, Artificial Sequence
  Description of Artificial Sequence Synthetic oligonucleotide motif sequence
ttctgaaacata

SEQ ID NO: 198 - DNA, 12 bp, Artificial Sequence
  Description of Artificial Sequence Synthetic oligonucleotide motif sequence
gcagtggtggaa

SEQ ID NO: 199 - DNA, 12 bp, Artificial Sequence
  Description of Artificial Sequence Synthetic oligonucleotide motif sequence
ttccaccactgc

SEQ ID NO: 200 - DNA, 12 bp, Artificial Sequence
  Description of Artificial Sequence Synthetic oligonucleotide motif sequence
atcactgggcag

SEQ ID NO: 201 - DNA, 12 bp, Artificial Sequence
  Description of Artificial Sequence Synthetic oligonucleotide motif sequence
ttgaaggctttg

SEQ ID NO: 202 - DNA, 12 bp, Artificial Sequence
  Description of Artificial Sequence Synthetic oligonucleotide motif sequence
ctcgcagtaagg

SEQ ID NO: 203 - DNA, 12 bp, Artificial Sequence
  Description of Artificial Sequence Synthetic oligonucleotide motif sequence
actctctgcatg

SEQ ID NO: 204 - DNA, 12 bp, Artificial Sequence
  Description of Artificial Sequence Synthetic oligonucleotide motif sequence
tctggagttatc

SEQ ID NO: 205 - DNA, 12 bp, Artificial Sequence
  Description of Artificial Sequence Synthetic oligonucleotide motif sequence
cttcgaacgctt

SEQ ID NO: 206 - DNA, 12 bp, Artificial Sequence
  Description of Artificial Sequence Synthetic oligonucleotide motif sequence
tactcaccaaat

SEQ ID NO: 207 - DNA, 12 bp, Artificial Sequence
  Description of Artificial Sequence Synthetic oligonucleotide motif sequence
ccttactgcgag

SEQ ID NO: 208 - DNA, 12 bp, Artificial Sequence
  Description of Artificial Sequence Synthetic oligonucleotide motif sequence
tacatgcagaga

SEQ ID NO: 209 - DNA, 12 bp, Artificial Sequence
  Description of Artificial Sequence Synthetic oligonucleotide motif sequence
agatggcagaaa

SEQ ID NO: 210 - DNA, 12 bp, Artificial Sequence
  Description of Artificial Sequence Synthetic oligonucleotide motif sequence
attgcagagact

SEQ ID NO: 211 - DNA, 12 bp, Artificial Sequence
  Description of Artificial Sequence Synthetic oligonucleotide motif sequence
caaggtctttgt

SEQ ID NO: 212 - DNA, 12 bp, Artificial Sequence
  Description of Artificial Sequence Synthetic oligonucleotide motif sequence
caaagccttcaa

SEQ ID NO: 213 - DNA, 12 bp, Artificial Sequence
  Description of Artificial Sequence Synthetic oligonucleotide motif sequence
gttatcagactt

SEQ ID NO: 214 - DNA, 12 bp, Artificial Sequence
  Description of Artificial Sequence Synthetic oligonucleotide motif sequence
atcaagcgcagt

SEQ ID NO: 215 - DNA, 12 bp, Artificial Sequence
  Description of Artificial Sequence Synthetic oligonucleotide motif sequence
tttgagcattac

SEQ ID NO: 216 - DNA, 12 bp, Artificial Sequence
  Description of Artificial Sequence Synthetic oligonucleotide motif sequence
gtaatgctcaaa

SEQ ID NO: 217 - DNA, 12 bp, Artificial Sequence
  Description of Artificial Sequence Synthetic oligonucleotide motif sequence
aagcgttcgaag

SEQ ID NO: 218 - DNA, 12 bp, Artificial Sequence
  Description of Artificial Sequence Synthetic oligonucleotide motif sequence
agagagcaccca

SEQ ID NO: 219 - DNA, 12 bp, Artificial Sequence
  Description of Artificial Sequence Synthetic oligonucleotide motif sequence
gataactccaga

SEQ ID NO: 220 - DNA, 12 bp, Artificial Sequence
  Description of Artificial Sequence Synthetic oligonucleotide motif sequence
agtctctgcaat

SEQ ID NO: 221 - DNA, 12 bp, Artificial Sequence
  Description of Artificial Sequence Synthetic oligonucleotide motif sequence
ttcgtcataagg

SEQ ID NO: 222 - DNA, 12 bp, Artificial Sequence
  Description of Artificial Sequence Synthetic oligonucleotide motif sequence
cgatgcgtaacc

SEQ ID NO: 223 - DNA, 12 bp, Artificial Sequence
  Description of Artificial Sequence Synthetic oligonucleotide motif sequence
aggtactctgca

SEQ ID NO: 224 - DNA, 12 bp, Artificial Sequence
  Description of Artificial Sequence Synthetic oligonucleotide motif sequence
tttctgccatct

SEQ ID NO: 225 - DNA, 12 bp, Artificial Sequence
  Description of Artificial Sequence Synthetic oligonucleotide motif sequence
atttggtgagta

SEQ ID NO: 226 - DNA, 12 bp, Artificial Sequence
  Description of Artificial Sequence Synthetic oligonucleotide motif sequence
ctacgctcctac

SEQ ID NO: 227 - DNA, 12 bp, Artificial Sequence
  Description of Artificial Sequence Synthetic oligonucleotide motif sequence
ggttagccatgc

SEQ ID NO: 228 - DNA, 12 bp, Artificial Sequence
  Description of Artificial Sequence Synthetic oligonucleotide motif sequence
actgcgcttgat

SEQ ID NO: 229 - DNA, 12 bp, Artificial Sequence
  Description of Artificial Sequence Synthetic oligonucleotide motif sequence
caaagcatctgg

SEQ ID NO: 230 - DNA, 12 bp, Artificial Sequence
  Description of Artificial Sequence Synthetic oligonucleotide motif sequence
tcaaagcatctg

SEQ ID NO: 231 - DNA, 12 bp, Artificial Sequence
  Description of Artificial Sequence Synthetic oligonucleotide motif sequence
ccagatgctttg

SEQ ID NO: 232 - DNA, 12 bp, Artificial Sequence
  Description of Artificial Sequence Synthetic oligonucleotide motif sequence
acttgtcccagc

SEQ ID NO: 233 - DNA, 13 bp, Artificial Sequence
  Description of Artificial Sequence Synthetic oligonucleotide motif sequence
ggtggtatctttg

SEQ ID NO: 234 - DNA, 13 bp, Artificial Sequence
  Description of Artificial Sequence Synthetic oligonucleotide motif sequence
acttctcttgcga

SEQ ID NO: 235 - DNA, 13 bp, Artificial Sequence
  Description of Artificial Sequence Synthetic oligonucleotide motif sequence
accaaagatacca

SEQ ID NO: 236 - DNA, 13 bp, Artificial Sequence
  Description of Artificial Sequence Synthetic oligonucleotide motif sequence
ccagatgctttga

SEQ ID NO: 237 - DNA, 13 bp, Artificial Sequence
  Description of Artificial Sequence Synthetic oligonucleotide motif sequence
tgttcttgctgaa

SEQ ID NO: 238 - DNA, 13 bp, Artificial Sequence
  Description of Artificial Sequence Synthetic oligonucleotide motif sequence
actgatggaaaac

SEQ ID NO: 239 - DNA, 13 bp, Artificial Sequence
  Description of Artificial Sequence Synthetic oligonucleotide motif sequence
aatctatcagcaa

SEQ ID NO: 240 - DNA, 13 bp, Artificial Sequence
  Description of Artificial Sequence Synthetic oligonucleotide motif sequence
aatcactgggcag

SEQ ID NO: 241 - DNA, 13 bp, Artificial Sequence
  Description of Artificial Sequence Synthetic oligonucleotide motif sequence
cttagcaatctgt

SEQ ID NO: 242 - DNA, 13 bp, Artificial Sequence
  Description of Artificial Sequence Synthetic oligonucleotide motif sequence
aaaggagacttaa

SEQ ID NO: 243 - DNA, 13 bp, Artificial Sequence
  Description of Artificial Sequence Synthetic oligonucleotide motif sequence
cattctctgattt

SEQ ID NO: 244 - DNA, 13 bp, Artificial Sequence
  Description of Artificial Sequence Synthetic oligonucleotide motif sequence
tttacatctcttc

SEQ ID NO: 245 - DNA, 13 bp, Artificial Sequence
  Description of Artificial Sequence Synthetic oligonucleotide motif sequence
tgttgagtatttc

SEQ ID NO: 246 - DNA, 13 bp, Artificial Sequence
  Description of Artificial Sequence Synthetic oligonucleotide motif sequence
cagatgctttgat

SEQ ID NO: 247 - DNA, 13 bp, Artificial Sequence
  Description of Artificial Sequence Synthetic oligonucleotide motif sequence
atactcaccaaat

SEQ ID NO: 248 - DNA, 13 bp, Artificial Sequence
  Description of Artificial Sequence Synthetic oligonucleotide motif sequence
cttaactcctgaa

SEQ ID NO: 249 - DNA, 13 bp, Artificial Sequence
  Description of Artificial Sequence Synthetic oligonucleotide motif sequence
tggtatctttggt

SEQ ID NO: 250 - DNA, 13 bp, Artificial Sequence
  Description of Artificial Sequence Synthetic oligonucleotide motif sequence
tattttaattgag

SEQ ID NO: 251 - DNA, 13 bp, Artificial Sequence
  Description of Artificial Sequence Synthetic oligonucleotide motif sequence
ttcagcaagaaca

SEQ ID NO: 252 - DNA, 13 bp, Artificial Sequence
  Description of Artificial Sequence Synthetic oligonucleotide motif sequence
ttgcttcaaagtt

SEQ ID NO: 253 - DNA, 13 bp, Artificial Sequence
  Description of Artificial Sequence Synthetic oligonucleotide motif sequence
gttttccatcagt

SEQ ID NO: 254 - DNA, 13 bp, Artificial Sequence
  Description of Artificial Sequence Synthetic oligonucleotide motif sequence
tgctgtggttgtt

SEQ ID NO: 255 - DNA, 13 bp, Artificial Sequence
  Description of Artificial Sequence Synthetic oligonucleotide motif sequence
tacatgcagagag

SEQ ID NO: 256 - DNA, 13 bp, Artificial Sequence
  Description of Artificial Sequence Synthetic oligonucleotide motif sequence
caaacccatcaag

SEQ ID NO: 257 - DNA, 13 bp, Artificial Sequence
  Description of Artificial Sequence Synthetic oligonucleotide motif sequence
atttggtgagtat

SEQ ID NO: 258 - DNA, 13 bp, Artificial Sequence
  Description of Artificial Sequence Synthetic oligonucleotide motif sequence
aaacccatcaagt

SEQ ID NO: 259 - DNA, 13 bp, Artificial Sequence
  Description of Artificial Sequence Synthetic oligonucleotide motif sequence
ctcaattaaaata

SEQ ID NO: 260 - DNA, 13 bp, Artificial Sequence
  Description of Artificial Sequence Synthetic oligonucleotide motif sequence
cttgatgggtttg

SEQ ID NO: 261 - DNA, 13 bp, Artificial Sequence
  Description of Artificial Sequence Synthetic oligonucleotide motif sequence
aaatcagagaatg

SEQ ID NO: 262 - DNA, 13 bp, Artificial Sequence
  Description of Artificial Sequence Synthetic oligonucleotide motif sequence
aacgttaatattc

SEQ ID NO: 263 - DNA, 13 bp, Artificial Sequence
  Description of Artificial Sequence Synthetic oligonucleotide motif sequence
aacaaccacagca

SEQ ID NO: 264 - DNA, 13 bp, Artificial Sequence
  Description of Artificial Sequence Synthetic oligonucleotide motif sequence
ttagagctttacg

SEQ ID NO: 265 - DNA, 13 bp, Artificial Sequence
  Description of Artificial Sequence Synthetic oligonucleotide motif sequence
acagattgctaag

SEQ ID NO: 266 - DNA, 13 bp, Artificial Sequence
  Description of Artificial Sequence Synthetic oligonucleotide motif sequence
atcaaagcatctg

SEQ ID NO: 267 - DNA, 13 bp, Artificial Sequence
  Description of Artificial Sequence Synthetic oligonucleotide motif sequence
caaagataccacc

SEQ ID NO: 268 - DNA, 13 bp, Artificial Sequence
  Description of Artificial Sequence Synthetic oligonucleotide motif sequence
ttaagtctccttt

SEQ ID NO: 269 - DNA, 13 bp, Artificial Sequence
  Description of Artificial Sequence Synthetic oligonucleotide motif sequence
acttgatgggttt

SEQ ID NO: 270 - DNA, 13 bp, Artificial Sequence
  Description of Artificial Sequence Synthetic oligonucleotide motif sequence
caattaactcacc

SEQ ID NO: 271 - DNA, 13 bp, Artificial Sequence
  Description of Artificial Sequence Synthetic oligonucleotide motif sequence
tcaaagcatctgg

SEQ ID NO: 272 - DNA, 13 bp, Artificial Sequence
  Description of Artificial Sequence Synthetic oligonucleotide motif sequence
gattttaccatta

SEQ ID NO: 273 - DNA, 13 bp, Artificial Sequence
  Description of Artificial Sequence Synthetic oligonucleotide motif sequence
gaagagatgtaaa

SEQ ID NO: 274 - DNA, 13 bp, Artificial Sequence
  Description of Artificial Sequence Synthetic oligonucleotide motif sequence
agaaccagaaagt

SEQ ID NO: 275 - DNA, 13 bp, Artificial Sequence
  Description of Artificial Sequence Synthetic oligonucleotide motif sequence
gaatattaacgtt

SEQ ID NO: 276 - DNA, 13 bp, Artificial Sequence
  Description of Artificial Sequence Synthetic oligonucleotide motif sequence
acatgcagagagt

SEQ ID NO: 277 - DNA, 13 bp, Artificial Sequence
  Description of Artificial Sequence Synthetic oligonucleotide motif sequence
gttcgtcataagg

SEQ ID NO: 278 - DNA, 13 bp, Artificial Sequence
  Description of Artificial Sequence Synthetic oligonucleotide motif sequence
aactttgaagcaa

SEQ ID NO: 279 - DNA, 13 bp, Artificial Sequence
  Description of Artificial Sequence Synthetic oligonucleotide motif sequence
tcgcaagagaagt

SEQ ID NO: 280 - DNA, 13 bp, Artificial Sequence
  Description of Artificial Sequence Synthetic oligonucleotide motif sequence
ttcaggagttaag

SEQ ID NO: 281 - DNA, 13 bp, Artificial Sequence
  Description of Artificial Sequence Synthetic oligonucleotide motif sequence
ctgcccagtgatt

SEQ ID NO: 282 - DNA, 13 bp, Artificial Sequence
  Description of Artificial Sequence Synthetic oligonucleotide motif sequence
actctctgcatgt

SEQ ID NO: 283 - DNA, 13 bp, Artificial Sequence
  Description of Artificial Sequence Synthetic oligonucleotide motif sequence
ccttatgacgaac

SEQ ID NO: 284 - DNA, 13 bp, Artificial Sequence
  Description of Artificial Sequence Synthetic oligonucleotide motif sequence
ttgctgatagatt

SEQ ID NO: 285 - DNA, 13 bp, Artificial Sequence
  Description of Artificial Sequence Synthetic oligonucleotide motif sequence
aatggtaaaatct

SEQ ID NO: 286 - DNA, 13 bp, Artificial Sequence
  Description of Artificial Sequence Synthetic oligonucleotide motif sequence
ggtgagttaattg

SEQ ID NO: 287 - DNA, 13 bp, Artificial Sequence
  Description of Artificial Sequence Synthetic oligonucleotide motif sequence
agattttaccatt

SEQ ID NO: 288 - DNA, 13 bp, Artificial Sequence
  Description of Artificial Sequence Synthetic oligonucleotide motif sequence
actttctggttct

SEQ ID NO: 289 - DNA, 13 bp, Artificial Sequence
  Description of Artificial Sequence Synthetic oligonucleotide motif sequence
gaaatactcaaca

SEQ ID NO: 290 - DNA, 13 bp, Artificial Sequence
  Description of Artificial Sequence Synthetic oligonucleotide motif sequence
cgtaaagctctaa

SEQ ID NO: 291 - DNA, 13 bp, Artificial Sequence
  Description of Artificial Sequence Synthetic oligonucleotide motif sequence
ctctctgcatgta

SEQ ID NO: 292 - DNA, 13 bp, Artificial Sequence
  Description of Artificial Sequence Synthetic oligonucleotide motif sequence
taatggtaaaatc

SEQ ID NO: 293 - DNA, 14 bp, Artificial Sequence
  Description of Artificial Sequence Synthetic oligonucleotide motif sequence
ccaaagataccacc

SEQ ID NO: 294 - DNA, 14 bp, Artificial Sequence
  Description of Artificial Sequence Synthetic oligonucleotide motif sequence
aaacccatcaagta

SEQ ID NO: 295 - DNA, 14 bp, Artificial Sequence
  Description of Artificial Sequence Synthetic oligonucleotide motif sequence
ggtggtatctttgg

SEQ ID NO: 296 - DNA, 14 bp, Artificial Sequence
  Description of Artificial Sequence Synthetic oligonucleotide motif sequence
gtggtatctttggt

SEQ ID NO: 297 - DNA, 14 bp, Artificial Sequence
  Description of Artificial Sequence Synthetic oligonucleotide motif sequence
actttctggttctc

SEQ ID NO: 298 - DNA, 14 bp, Artificial Sequence
  Description of Artificial Sequence Synthetic oligonucleotide motif sequence
acttctcttgcgat

SEQ ID NO: 299 - DNA, 14 bp, Artificial Sequence
  Description of Artificial Sequence Synthetic oligonucleotide motif sequence
accaaagataccac

SEQ ID NO: 300 - DNA, 14 bp, Artificial Sequence
  Description of Artificial Sequence Synthetic oligonucleotide motif sequence
cttgatgggtttga

SEQ ID NO: 301 - DNA, 14 bp, Artificial Sequence
  Description of Artificial Sequence Synthetic oligonucleotide motif sequence
aacgttaatattca

SEQ ID NO: 302 - DNA, 14 bp, Artificial Sequence
  Description of Artificial Sequence Synthetic oligonucleotide motif sequence
atcaaagcatctgg

SEQ ID NO: 303 - DNA, 14 bp, Artificial Sequence
  Description of Artificial Sequence Synthetic oligonucleotide motif sequence
agattttaccatta

SEQ ID NO: 304 - DNA, 14 bp, Artificial Sequence
  Description of Artificial Sequence Synthetic oligonucleotide motif sequence
tcaaacccatcaag

SEQ ID NO: 305 - DNA, 14 bp, Artificial Sequence
  Description of Artificial Sequence Synthetic oligonucleotide motif sequence
ctaatggtaaaatc

SEQ ID NO: 306 - DNA, 14 bp, Artificial Sequence
  Description of Artificial Sequence Synthetic oligonucleotide motif sequence
caattaactcacca

SEQ ID NO: 307 - DNA, 14 bp, Artificial Sequence
  Description of Artificial Sequence Synthetic oligonucleotide motif sequence
atcgcaagagaagt

SEQ ID NO: 308 - DNA, 14 bp, Artificial Sequence
  Description of Artificial Sequence Synthetic oligonucleotide motif sequence
tgttgagtatttct

SEQ ID NO: 309 - DNA, 14 bp, Artificial Sequence
  Description of Artificial Sequence Synthetic oligonucleotide motif sequence
ccagatgctttgat

SEQ ID NO: 310 - DNA, 14 bp, Artificial Sequence
  Description of Artificial Sequence Synthetic oligonucleotide motif sequence
gagaaccagaaagt

SEQ ID NO: 311 - DNA, 14 bp, Artificial Sequence
  Description of Artificial Sequence Synthetic oligonucleotide motif sequence
tacttgatgggttt

SEQ ID NO: 312 - DNA, 14 bp, Artificial Sequence
  Description of Artificial Sequence Synthetic oligonucleotide motif sequence
actctctgcatgta

SEQ ID NO: 313 - DNA, 14 bp, Artificial Sequence
  Description of Artificial Sequence Synthetic oligonucleotide motif sequence
gattttaccattag

SEQ ID NO: 314 - DNA, 14 bp, Artificial Sequence
  Description of Artificial Sequence Synthetic oligonucleotide motif sequence
tgaatattaacgtt

SEQ ID NO: 315 - DNA, 14 bp, Artificial Sequence
  Description of Artificial Sequence Synthetic oligonucleotide motif sequence
tacatgcagagagt

SEQ ID NO: 316 - DNA, 14 bp, Artificial Sequence
  Description of Artificial Sequence Synthetic oligonucleotide motif sequence
acttagcaatctgt

SEQ ID NO: 317 - DNA, 14 bp, Artificial Sequence
  Description of Artificial Sequence Synthetic oligonucleotide motif sequence
acagattgctaagt

SEQ ID NO: 318 - DNA, 14 bp, Artificial Sequence
  Description of Artificial Sequence Synthetic oligonucleotide motif sequence
taatggtaaaatct

SEQ ID NO: 319 - DNA, 14 bp, Artificial Sequence
  Description of Artificial Sequence Synthetic oligonucleotide motif sequence
tggtgagttaattg

SEQ ID NO: 320 - DNA, 14 bp, Artificial Sequence
  Description of Artificial Sequence Synthetic oligonucleotide motif sequence
gaattgcagaaagc

SEQ ID NO: 321 - DNA, 14 bp, Artificial Sequence
  Description of Artificial Sequence Synthetic oligonucleotide motif sequence
gctttctgcaattc

SEQ ID NO: 322 - DNA, 15 bp, Artificial Sequence
  Description of Artificial Sequence Synthetic oligonucleotide motif sequence
tgaattgcagaaagc

SEQ ID NO: 323 - DNA, 15 bp, Artificial Sequence
  Description of Artificial Sequence Synthetic oligonucleotide motif sequence
ctaatggtaaaatct

SEQ ID NO: 324 - DNA, 15 bp, Artificial Sequence
  Description of Artificial Sequence Synthetic oligonucleotide motif sequence
acttctcttgcgatt

SEQ ID NO: 325 - DNA, 15 bp, Artificial Sequence
  Description of Artificial Sequence Synthetic oligonucleotide motif sequence
aatcgcaagagaagt

SEQ ID NO: 326 - DNA, 15 bp, Artificial Sequence
  Description of Artificial Sequence Synthetic oligonucleotide motif sequence
tgttgagtatttctt

SEQ ID NO: 327 - DNA, 15 bp, Artificial Sequence
  Description of Artificial Sequence Synthetic oligonucleotide motif sequence
gctttctgcaattca

SEQ ID NO: 328 - DNA, 15 bp, Artificial Sequence
  Description of Artificial Sequence Synthetic oligonucleotide motif sequence
aagaaatactcaaca

SEQ ID NO: 329 - DNA, 15 bp, Artificial Sequence
  Description of Artificial Sequence Synthetic oligonucleotide motif sequence
agattttaccattag

SEQ ID NO: 330 - DNA, 15 bp, Artificial Sequence
  Description of Artificial Sequence Synthetic oligonucleotide motif sequence
ggtggtatctttggt

SEQ ID NO: 331 - DNA, 15 bp, Artificial Sequence
  Description of Artificial Sequence Synthetic oligonucleotide motif sequence
accaaagataccacc

SEQ ID NO: 332 - DNA, 15 bp, Artificial Sequence
  Description of Artificial Sequence Synthetic oligonucleotide motif sequence
ttacttgatgggttt

SEQ ID NO: 333 - DNA, 15 bp, Artificial Sequence
  Description of Artificial Sequence Synthetic oligonucleotide motif sequence
aaacccatcaagtaa

(19) United States
(12) Patent Application Publication
(10) Pub. No.: US 2007/0054357 A1
(43) Pub. Date: Mar. 8, 2007

(54) Identification of useful bacteriophage

(76) Inventors: Gary R. Pasternack, Baltimore, MD (US); Alexander Sulakvelidze, Baltimore, MD (US)

Correspondence Address: Bell, Boyd & Lloyd LLC, P.O. Box 1135, Chicago, IL, 60690-1135, US

(21) Appl. No.: 11/220,076

(22) Filed: Sep. 6, 2005

(51) Int. Cl. C12P 21/06

(52) U.S. Cl. 435/69.1

(57) ABSTRACT

A method of identifying particular bacteriophage is provided.

### FIELD OF THE INVENTION

This invention is directed to a method of identifying classes of bacteriophage useful for the control of, for example, Listeria monocytogenes and Salmonella species in environmental, food, medical, veterinary, agricultural, and other settings. More specifically, groups of nucleotide sequences are provided that are present in and identify a class of bacteriophages useful in the control of, for example, Listeria monocytogenes. Likewise, groups of short nucleotide sequences are provided that identify a class of bacteriophages useful in the control of, for example, Salmonella species. The field of the invention is restricted to bacteriophage genomes; the claimed sequences or group of sequences may occur in the genomes of organisms other than bacteriophages without prejudice to the present invention.

### REVIEW OF RELATED ART

There are six major families of bacteriophages including Myoviridae (T-even bacteriophages), Styloviridae (Lambda bacteriophage groups), Podoviridae (T-7 and related bacteriophage), Microviridae (X174 group), Leviviridae (for example, E coli bacteriophage MS2) and Inoviridae as well as coliphages, in general. Other bacteriophage families include members of the Cystoviridae, Microviridae, and Siphoviridae families.

Bacteriophage has been used therapeutically since the early part of the last century. Bacteriophage, which derive their name from the Greek word “phago” meaning “to eat” or “bacteria eaters”, were independently discovered by Twort as well as by D'Herelle in the first part of the twentieth century. Early enthusiasm led to the use of bacteriophage as both prophylaxis and therapy for diseases caused by bacteria. However, the results from early studies to evaluate bacteriophage as antimicrobial agents were variable due to the uncontrolled study design and the inability to standardize reagents. Later, in better designed and controlled studies, it was concluded that bacteriophage were not useful as antimicrobial agents (Pyle, N. J., J. Bacteriol, 12:245-61 (1936); Colvin, M. G., J. Infect. Dis., 51:17-29 (1932); and Boyd et al., Trans R. Soc. Trop. Med. Hyg., 37:243-62 (1944)).

This initial failure of phage as antibacterial agents may have been due to the failure to select for phage that demonstrated high in vitro lytic activity prior to in vivo use. For example, the phage employed may have had little or no activity against the target pathogen, or they may have been used against bacteria that were resistant due to lysogenization or the phage itself may have been lysogenic for the target bacterium (Barrow et al., Trends in Microbiology, 5:268-71 (1997)). However, with better understanding of the phage-bacterium interaction and of bacterial virulence factors, it has been possible to conduct studies which demonstrated the in vivo anti-bacterial activity of the bacteriophage (Asheshov et al., Lancet, 1:319-20 (1937); Ward, W. E., J. Infect. Dis., 72:172-6 (1943); and Lowbury et al., J. Gen. Microbiol., 9:524-35 (1953)). In the U.S. during the 1940's, Eli Lilly Co. commercially manufactured six phage products for human use, including preparations targeted towards Staphylococci, Streptococci and other respiratory pathogens.

With the advent of antibiotics, the therapeutic use of phage gradually fell out of favor in the U.S. and Western Europe, and little subsequent research was conducted. However, in the 1970's and 1980's bacteriophage therapy continued to be utilized in Eastern Europe, most notably in Poland and the former Soviet Union. Alisky et al. conducted a review of all Medline citations where bacteriophage was employed therapeutically from 1966 to 1996 (Alisky et al., J. Infect., 36:5-15 (1998)).

There are also several British studies describing controlled trials of bacteriophage raised against specific pathogens in experimentally infected animal models such as mice and guinea pigs (see, e.g., Smith, H. W. & M. B. Huggins, J. Gen. Microbiol. 128:307-318 (1982); Smith, H. W. & M. B. Huggins, J. Gen. Microbiol, 129:2659-2675 (1983); Smith, H. W. & R. B. Huggins, J. Gen. Microbiol., 133:1111-1126 (1987); and Smith et al., J. Gen. Microbiol., 133:1127-1135 (1987)). These trials measured objective criteria such as survival rates. Efficacy against Staphylococcus, Pseudomonas and Acinetobacter infections were observed. These studies are described in more detail below.

One such study concentrated on improving bioavailability of phage in live animals by modifying the bacteriophage (Merril et al., Proc. Natl. Acad. Sci. USA, 93:3188-3192 (1996)). Reports from the U.S. relating to bacteriophage administration for diagnostic purposes have indicated phage have been safely administered to humans to monitor humoral immune response in adenosine deaminase deficient patients (Ochs et al., Blood, 80:1163-71 (1992)) and for analyzing the importance of cell-associated molecules in modulating the immune response in humans (Ochs et al., Clin. Immunol. Immunopathol., 67:S33-40 (1993)).

Additionally, Polish, Georgian and Russian papers describe experiments where phage was administered systemically, topically or orally to treat a wide variety of antimicrobial resistant pathogens (see, e.g., Shabalova et al., Abstr. 443. In Proceedings of IX International Cystic Fibrosis Congress, Dublin, Ireland; Slopek et al., Archivum. Immunol. Therapiae Experimental, 31:267-291 (1983); and Slopek et al., Archivum Immunol. Therapiae Experimental, 35:569-83 (1987)).

Infections treated with bacteriophage included osteomyelitis, sepsis, empyema, gastroenteritis, suppurative wound infection, pneumonia and dermatitis. Pathogens treated with the bacteriophage include Staphylococci, Streptococci, Klebsiella, Shigella, Salmonella, Pseudomonas, Proteus and Escherichia. Articles have reported a range of success rates for phage therapy between 80-95% with only rare reversible allergic or gastrointestinal side effects. These results indicate that bacteriophage may be a useful adjunct in the fight against bacterial diseases.

Despite the use of bacteriophage for the treatment of diseases in humans, there remains in the art a need for the discovery of novel bacteriophage and methods for using these bacteriophage in several critical areas. One significant need concerns the treatment of processed or unprocessed food products to treat or prevent colonization with undesirable pathogens such as Listeria monocytogenes or Salmonella which are responsible for food-borne illness. A second critical area of need concerns the removal of undesirable bacteria from industrial environments such as food processing facilities to prevent colonization thereof. A third critical area of need concerns the removal of undesirable pathogens such as L. monocytogenes and Salmonella from environments where they may be passed to susceptible humans and animals, such as supermarkets, hospitals, nursing homes, veterinary facilities, and other such environments. Finally, new bacteriophage and methods of using the same are needed for the treatment of human or animal bacterial disease.

The present invention provides nucleic acid sequences that uniquely define useful classes of bacteriophage. These nucleic acid sequences are termed oligonucleotide motifs. The scientific literature does not directly address notion that specific amino acid or nucleic acid motifs identify groups of bacteriophage specific for specific commercially or medically important bacterial pathogens. Blaisdell (Blaisdell et al. Similarities and dissimilarities of phage genomes, Proceedings of the National Academy of Sciences of the United States of America, 93, 5854 (1996)) recognized that the genomes of bacteriophages contain short oligonucleotide signatures that can be used to construct a taxonomy of bacteriophages and show their relatedness to one another. Blaisdell and colleagues focused upon di- and tetra-nucleotides that were not related to host range in any systematic fashion.

In other studies, some degree of homology has been noted at a crude level, however not at the level of sequences of amino acids or nucleotides that would provide guidance to the skilled practitioner. Salgado (1) studied the homology between two individual bacteriophages, Salmonella enterica serovar Typhimurium phage P22 and Salmonella enterica serovar Anatum var. 15+ phage ε³⁴. Using DNA restriction digest patterns, reaction of both phages with antibodies raised to the P22 phage, and the common reactivity of the tailspike proteins with a monoclonal antibody as evidence, the authors concluded that there significant homology between these phages. Since the tailspike proteins are thought to react with the lipopolysaccharide (LPS) of the two Salmonella serovars, the authors concluded that further studies would be required to establish a role for their findings in determining the specificity of the phage. Although the common reaction with a monoclonal antibody provided evidence of an epitope shared between the tailspike proteins, the studies did not demonstrate the absence of this degree of homology, nor the absence of the monoclonal reactivity in phages that failed to react with Salmonella species.

The highly variable protein sequence near the tip of the long tail fiber proteins in T-even phages encodes the adhesins that determine the ability of the phage to bind to specific bacterial hosts according to the studies of Tetart (2). These studies compared the sequences of the adhesins in the distal tail fibers of T-even phage, finding that recombination in this restricted area led to a change in specificity of the adhesins, and, hence, a change in host range.

Certain hosts require that bacteriophage utilize hyaluronidases in order to effect entry. Marciel (3) compared sequences of hyl (hyaluronidase) genes from 13 bacteriophage specific for Streptococcus pyogenes. These investigators noted allelic variation, where the hyl gene from some strains included a motif of a collagen-like domain, and others did not. The studies did not draw any conclusions regarding the effects of allelic variation on host range.

Loessner et al. (4) carried out important studies of murein hydrolases of phage specific for Listeria monocytogenes. Murein hydrolases are enzymes involved in the lysis of the host bacterial cell after phage replication has occurred. Using sequences derived from two phage specific for Listeria monocytogenes, Loessner and colleagues proposed a modular organization of motifs within these enzymes that would, in fact, facilitate a broad host range through ready utilization of pre-existing catalytic and cell wall binding domains in response to changing conditions. The Loessner studies only addressed the lytic phase of bacteriophage infection and did not, except as noted, address issues of host range, since host range is critically determined by the initial attachment of a bacteriophage to a bacterium, and not by lysis.

Chua (5) published a detailed study of the tailspike protein of the lysogenic Shigella flexneri bacteriophage Sf6. The studies focused upon correlating specific motifs with catalytic activity, and relating the specific catalytic activities to the ability to cleave or otherwise modify specific bacterial antigens. The study did not, however, attempt to relate the presence or absence of specific motifs to host range.

Chipman (6) used X-ray diffraction to study similarities of the capsid proteins of the Spirolasma melliferum phage SpV4 to the Chlamydia phage, Chp1, and the coliphages alpha 3, phi K, G4 and phi X174. These studies identified a hydrophobic cavity that they speculated might serve as a common receptor recognition site during host infection. The study did not develop any information concerning motifs that might govern host range.

Some studies address the attachment of bacteriophage integrases to the host genome. Integration is a feature limited to so-called temperate, or lysogenic, bacteriophages. Although motifs found in integrases could conceivably correlate with host range, they are not relevant to this application since lysogenic bacteriophages are expressly excluded from the subject matter of this invention. Examples of studies using sequence motifs to focus on the mechanism of integration of lysogenic bacteriophages include Dorgai (7), Kaneko (8) and Salmi (9).

Crutz-Le Coq and colleagues (10) obtained the complete sequence of the 31754 bp genome of bIL170, a virulent bacteriophage of Lactococcus lactis belonging to the 936 group. Analysis of this sequence identified a 110 to 150 amino acid hypervariable region flanked by conserved 20 amino acid sequences. The authors hypothesized that this sequence could encode molecules involved in host range determination, however no specific attempt was made to distinguish common motifs among phage lytic for a given bacterial strain or species. Meanwhile, Gottlieb (11, 12) sequenced the genome of phi12, a phage related to phi6, solely for purposes of speciation without addressing host range or specificity, save to note a similarity of the phi12 attachment proteins to those of phi13. While Crutz-Le Coq extensively discusses the potential role of sequence variations in specific genes as determinants of host range, there is no anticipation of sequence motifs that would define useful groups of bacteriophage on the basis of specific, defining sequence motifs. Similar approaches include those of Tu (13), who sequenced the mycoplasma P1 genome and assigned provisional functions on the basis of sequence motifs. Weisberg (14) sequenced the lysogenic filamentous phage HK022, and used the sequence information in an evolutionary context to compare strategies developed by phage to deal with similar problems. Likewise, Pfister (15) sequenced psiM2, focusing upon structure-function assignments and sequence comparisons aimed at establishing the evolutionary hierarchy.

In examining bacteriophage phiKZ as a candidate therapeutic phage for Pseudomonas aeruginosa, Mesyanzhinov et al. (16) obtained its complete DNA sequence. Analysis of this phage included identification of the individual genes and identification of motifs common to many phages. The results bolstered the understanding that many phage are derived from common ancestors, but did not identify sequences or motifs potentially involved in determination of host range.

Altieri (17) described a NusB contains a 10 residue Arg-rich RNA-binding motif (ARM) at the N-terminus but is not sequentially homologous to any other proteins. This motif was used to show that this particular lambda protein, NusB, involved in transcriptional control is, through its structure, a member of a class of alpha-helical RNA-binding proteins.

Verheust (18) noted that in tectiviruses, an unusual phage group whose double stranded DNA lies within a lipid vesicle inside a protein coat, those organisms infecting gram-negative bacteria are closely related. Focusing upon tectiviruses infecting gram positive bacteria, these authors found that mutations in a particular motif in GIL01 and GIL16 phages correlate with a switch to a lytic cycle from a temperate cycle. Both bacterial viruses displayed narrow, yet slightly different, host spectrums.

Akulenko (19) focused upon motifs to define by analogy evolutionarily conserved sequences in the catalytic sites of enzymes. Cannistraro examined protein-level structure-function relationships in bacteriophage T7 DNA polymerase (20). Likewise, Lebars (21) explored structural motifs as part of determining the mechanism of action of the T4 RegB endonuclease, building upon the prior functional studies of Sanson (22). Meanwhile Lee (23) determined motifs comprised of critical lysine residues required for the function of the RNA polymerase domain of bacteriophage T7 helicase-primase. Other work, e.g. that of Benevides (24) has focused upon the common structural features of phage capsid proteins that permit their self assembly. Other studies of motifs common among capsid proteins include those of Pederson (25). Kim (26) looked at a motif in the Nun protein of prophage HK022 that was responsible for the exclusion of superinfection by other phage such as lambda. Many papers deal with common structural motifs in encoded proteins that contribute to their function. Among these are the studies of Kumaraswami (27) examining features of the Mor/C family of transcriptional activators in bacteriophage Mu, and Li (28) examining the transcriptional activation domain of the T4 protein MotA.

Some motifs identify sequences encoding catalytically active nucleic acids. An example is that of Lindqvist (29) of the T4 nrdB group I intron likely encoding a ribozyme.

Certain motifs appear in proteins of temperate, or lysogenic phages, that are involved in the mechanism of lysogeny. Motifs occurring in these proteins have been studied with the goal of clarifying the mechanism of lysogeny. These studies are not directed toward defining useful class of bacteriophage. Examples of such studies include Rutkai (30).

Other studies of motifs are directed toward gaining a better understanding of how bacteriophages function. Studies using motifs to understand so-called immunity, whereby phage-infected bacteria are resistant to superinfection by another bacteriophage, include those of Defenbaugh (31) and Stuart (32). Mitchell (33) examined how bacteriophage DNA is loaded into the empty capsids during replication; he found that the enzymes that accomplish this, bacteriophage terminases, contain Walker A motifs that are signatures for ATPase catalytic sites. Other studies identifying motifs playing a role in packaging of bacteriophage nucleic acids are those of Benevides (34), Brunel (35), Mitchell (36), Rao (37), Rodriguez-Casado (38), Kuebler (39), Parker (40), Tuma (41) and Tao (42).

Other investigators sought to identify motifs signifying enzymes involved in replication of phage DNA or DNA repair, such as DNA polymerases and polynucleotide kinases, as well as enzymes involved in excision of temperate phages and DNA methylation. Others examined phosphatases, such as the studies of White (43). Studies of motifs involved in replication include those of Eisenbrandt (44), Galburt (45), Imburgio (46), Karpel (47), Lee (48), Petrov (49), Rezende (50), Sam (51), Wojciak (52), Yeo (53), Bravo (54), Moyer (55), Radlinska (56), Schneider (57), Valentine (58), de Vega (59), Hoogstraten (60), Makeyev (61), Moscoso (62), Tseng (63) and Illana (64).

Although usually thought to be a feature of eukaryotic genomes, some bacteriophage genes may contain introns. For example, the Brussow laboratory (65-67) found that half of Streptococcus thermophilus phage examined contained a group IA2 intron in a lysin gene; this intron was associated with splicing of phage mRNA. A 14 base pair motif in the coding sequence was positively associated with the presence of an intron. Such motifs are useful in predicting whether a given gene will possess an intron, but are not useful in predicting the host range or other biologic properties of a bacteriophage.

Similar work has been carried out examining common motifs required for the function of proteins involved in translation. Examples include the studies of Sengupta (68).

Looking at phage gene transcription, Nechaev (69) found 8 to 10 base pair motifs that are involved in initiating transcription from T4 late promoters. In related studies, Orsini (70) examined the interaction of this same motif with a transcriptional inhibitory factor. Transcription is also regulated in T4 phage by a somewhat divergent family of RegB endonucleases (71) that cleave and process phage RNA's through specific tetranucleotide motifs. Vieu (72) applied similar approaches to identify bacteriophage genetic elements controlling termination of transcription in lambda phage. Additional papers addressing transcriptional control of bacteriophage genes through identification of motifs include Christie (73), Cilley (74), Fromknecht (75), Kim (76), Marshall-Batty (77), Mukhopadhyay (78), Paul (79), Ho (80), Li (81), Pande (82), Urbauer (83), Faber (84), Scharpf (85) and Watnick (86).

Protein motifs are also important in bacteriophage assembly. Bernal (87), for example, specifically focused upon the role of protein folding motifs in the self assembly of bacteriophage alpha3. Other studies dealing specifically with the role of protein motifs in phage assembly include Rentas (88).

Bleuit (89) looked for a conserved motif in the UvsY protein in T4 bacteriophage that correlated with its DNA binding activity as a recombination mediator protein. These investigators studied how modification of the motif structure influenced its function. Melnyk (90) used motifs in the M13 major coat protein to study how specific protein sequences facilitate low affinity dimer formation. In other structural work, Papanikolopoulou (91, 92) looked at protein folding motifs in bacteriophage adhesins. The studies of Qu and colleagues (93) are similar in that they examined the role of coiled-coil motifs in bacteriophage tail fiber assembly. Van Raaij (94) determined the crystal structure of the T4 proteins tail fiber required for adhesion to its E. coli host. This study correlated structure with function, but did not identify motifs that would correlate with host range or specificity. Likewise, Sam (95) used X-ray diffraction to identify a winged helix motif important for the function of the Xis excisionase in bacteriophage lambda, and Knowlton (96) studied NusG structure, since it is a highly conserved protein linked to termination in many prokaryotic species.

A number of studies are purely genetic in nature, focused upon elaboration of structure-function relationships through comparison of sequences of different bacteriophage. For instance, Sau (97) compared sequences of repressor genes of temperate mycobacteriophage L1 with a mutant gene and other mycobacteriophage repressor proteins; these studies were directed solely toward a better understanding of repressor protein function. Other studies of repressors include those of Shearwin (98). In related studies, Lanthaler (99) localized the gene encoding an endonuclease to a phage introns, then further identified sequence motifs shared with other endonucleases. Other substantially genetic studies include those of Lee (100).

Some studies focused upon phage proteins with specific enzymatic motifs, and used variations among such proteins to gain a better understanding of the function of that enzyme. Examples include the studies of Reiter (101), Zhu (102), Chan (103), Goetzinger (104), Lin (105), Logan (106), Rodriguez (107), Rogov (108), Wang (109) and Yin (110).

Other studies, such as that of Romero (111), identified close homologues of bacterial LytA amidases in two lysogenic phage of Streptococcus mitis; structural and functional comparison of the phage enzymes with the bacterial version permitted assignment of specific functions to specific amino acid residues. In related work, Calin-Jageman identified structural RNA motifs that served to inhibit certain bacterial polymerases (112).

For purely technical purposes, Chan (113) used repeated 7- and 8-base nucleic acid motifs within lambda DNA to validate a novel DNA mapping technology. This work did not test for the presence or functional significance of these motifs across different phage.

Interestingly, Dabrowska (114) examined interactions between phages and various eukaryotic cells, observing binding of phages to the membranes of cancer and normal blood cells. Wild-type phage T4 (wtT4) and its substrain HAP1 with enhanced affinity for melanoma cells inhibit markedly and significantly experimental lung metastasis of murine B16 melanoma cells by 47% and 80%, respectively. A possible molecular mechanism of these effects, namely a specific interaction between the Lys-Gly-Asp motif of the phage protein 24 and beta3-integrin receptors on target cells is proposed. It was also shown that anti-beta3 antibodies and synthetic peptides mimicking natural beta3 ligands inhibit the phage binding to cancer cells. This is in line with the well-described beta3 integrin-dependent mechanism of tumor metastasis. It is concluded that the blocking of beta3 integrins by phage preparations results in a significant decrease in tumor invasiveness.

Doulatov (115) examined the ability of Bordetella phage to generate diversity in a gene specifying host tropism—the gene is a reverse transcriptase. Using the Bordetella phage cassette as a signature, they identified numerous related elements in diverse bacteria. These elements constitute a new family of retroelements with the potential to confer selective advantages to their host genomes.

Some papers deal with both motifs and heterogeneity in the gene cassette encoding the holins and lysins required for bacterial lysis. In this regard, Labrie (116) examined heterogeneity in the lysis cassette of Lactococcus lactis phages, with specific reference to the bacteriophage u136 holin. These studies focused specifically upon the presence of motifs encoding specific enzymatic activities, such as amidases or muramidases, but did not address the issue of which activities might confer specificity. Vukov (117, 118) studied control and structure-function relationships in the Listeria monocytogenes bacteriophage A118 holin, but did not address how this contributed to the specificity of this phage for Listeria. Barenboim noted that some holin genes possess motifs that encode two distinct translational start sites, which in turn yield two distinct holins from the same gene (119).

Some work in the literature is directed toward the introduction of non-native motifs into phage to create improved vectors for gene therapy. Examples of this include the work of Piersanti (120).

### SUMMARY

Oligonucleotides common to a selected group of bacteriophage can be used to screen new bacteriophage to determine whether the new bacteriophage shares in the specificity as the selected group of bacteriophage.

The invention relates to a method using said oligonucleotides to identify bacteriophage of interest.

The invention relates to an isolated bacteriophage comprising at least two of said oligonucleotides.

Additional features and advantages are described herein, and will be apparent from, the following Detailed Description.

### DETAILED DESCRIPTION

The present invention relates to the use of nucleotide sequences to identify classes of bacteriophages useful in controlling or eliminating bacterial pathogens from environmental, food, medical, veterinary, agricultural, and other settings.

At least three type strains of a particular bacterium which is a lytic target of a bacteriophage are selected to comprise a bacterium test data set. A candidate phage then is tested for lytic activity in all of the strains of the bacterium test data set. As controls, bacteria of a related strain, another species or another genus can be used. Preferably, the bacterium test data set comprises at least 4, 5, 6, 7, 8, 9, 10 or more strains of a bacterium of interest.

As the target of the phage taught herein, any bacterium can be used, including, for example, bacteria of the genus Pseudomonas, Clostridium, Enterobacter, Propionibacter, Vibrio, Xanthomonas, Mycoplasma, Acinetobacter, Chlamydia, Acetobacter, Aeromonas, Agrobacterium, Alcaligenes, Anabena, Archaebacteria, Azotobacter, Bacillus, Borrelia, Campylobacter, Citrobacter, Corynebacterium, Cyanobacteria, Desulfovibrio, Enterococcus, Erwinia, Escherichia, Flavobacterium, Hemophilus, Klebsiella, Lactobacillus, Listeria, Mycobacterium, Mycococcus, Pasteurella, Proteus, Rhodobacter, Salmonella, Shigella, Serratia, Staphylococcus, Streptococcus, Streptomyces, Thermus, Yersinia, Actinomyces, Brucella, Lactococcus, Brevibacterium, Clavibacter, Halobacterium, Helicobacter and so on. Type strains can be obtained from the ATCC or can be obtained practicing methods known in the art.

One embodiment comprises a composition of a bacteriophage whose genome contains two or more sequences drawn from the list comprising Table 1. The phage of interest may contain at least 3, at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 of the oligonucleotides of interest. This composition of bacteriophages comprises a group of lytic bacteriophages whose host range is specific for Listeria monocytogenes. The bacteriophage in the composition may contain one copy of sequence drawn from the list in Table 1, or it can contain two or more copies of such sequences. A preferred embodiment comprises a composition of two or more genetically distinct bacteriophages, each of whose genomes contains two or more sequences drawn from the list comprising Table 1. In this embodiment, the genome of each bacteriophage in the composition may contain the same sequences drawn from the list in Table 1 as any other bacteriophage in the composition, or it may differ in one, more than one, or all sequences drawn from the list in Table 1. The genomes of bacteriophages in this composition may contain the same number of sequences drawn from the list in Table 1, or they may contain different numbers of such sequences. The genome of each bacteriophage in the composition may contain one copy of sequence drawn from the list in Table 1, or it can contain two or more copies of such sequences.

As used herein, “complement” is meant to indicate a second oligonucleotide that hybridizes to a first oligonucleotide. Thus, if a first oligonucleotide has a sequence ATGC, the complement is TACG. As used herein, “reverse complement” is a second oligonucleotide that hybridizes to a first oligonucleotide taking into account the polarity of the strand, the first oligonucleotide, ATGC presented in the 5′ to 3′ direction, and the reverse complement also presented in the 5′ to 3′ direction and thus would be GCAT.

Lytic phage are expanded clonally as known in the art. Specificity for a bacterium of interest is ascertained practicing methods known in the art. The genome of the phage of interest is obtained practicing methods known in the art.

For each group of genomic bacteriophage sequences, a set of oligonucleotides was computed such that: [1] each oligonucleotide was 3 nucleotides or longer; [2] each oligonucleotide was as long as possible; [3] an oligonucleotide could hybridize to either strand of the bacteriophage genomic sequence; [4] every oligonucleotide was present in every member of the defining group; and [5] no oligonucleotide was present in any member of the other group. As used herein, an oligonucleotide that satisfies at least conditions [1], [3] and [4], and preferably [1], [3]. [4] and [5], is also known as a “motif.” A “motif set” comprises at least two motifs, at least 3, at least 4, at least 5, at least 6, at least 7, at least 8, at least 9, at least 10, at least 11, at least 12, at least 13, at least 14, at least 15 or more motifs.

Operationally, the first three criteria were met in the following manner:

Step 1. For each phage genome sequence, the position x was set such that x=1 on the plus strand.

Step 2. The oligonucleotide of length L=3 commencing at position x was stored.

Step 3. x was sequentially incremented by 1 nucleotide from x=1 to x=n, where n equals the length of the phage genome.

Step 4. Step 2 was repeated at each position from x=2 to x=n.

Step 5. The oligonucleotide length L was then incremented by 1 and Steps 1 through 4 were repeated.

Step 6. Step 5 was repeated for each length L from L=2 through L=n.

Step 7. Step 1 through Step 6 were repeated except that Step 1 was modified to set the position x to x=1 on the minus strand.

Step 8. The resultant set of oligonucleotides varying in length from L=3 to L=n were then compared to each member of the defining group (e.g. specific for Listeria monocytogenes or Salmonella sp.). The presence of the exact oligonucleotide sequence and the number of occurrences on either the plus or minus strand were recorded.

Step 9. Oligonucleotides occurring in the sequence of each member of the defining group were retained, and those oligonucleotides failing to occur in every member of the defining group were discarded.

Step 10. The set of oligonucleotide motifs remaining after Step 9 were each individually tested for exact occurrence in a set of 407 phage genomes representing the majority of currently known phage genomes as described.

Step 11. Oligonucleotide motifs occurring in any phage sequence other than that of a bacteriophage belonging to the defining group (e.g. specific for Listeria monocytogenes or Salmonella sp.) were discarded. Only oligonucleotide motifs occurring only in the defining group were retained.

The procedures outlined in Step 1 through Step 11 can be accomplished by computational means well-known to those skilled in the art. The methods can range from simple manual string searches to use of more sophisticated homology search algorithms such as BLAST, provided that the search parameters are adjusted to retain short exact matches as significant.

An oligonucleotide of interest is one that is found at least once in the genome of each of the at least three species-specific, lytic phage of interest that comprise the phage test data set. The phage test data set can comprise at least four, five, six, seven, eight, nine, ten, eleven, twelve, thirteen, fourteen, fifteen or more species-specific, lytic phage strains.

A new candidate bacteriophage then is tested for presence of the two or more oligonucleotides by means of detecting including, but are not limited to: [1] isolation of the bacteriophage genome in its entirety or in any sub-portion followed by DNA sequencing by any means including but not limited to dideoxy sequencing, chemical sequencing according to Gilbert and Maxam, and sequencing by mass spectrometry; [2] polymerase chain reaction (PCR) whereby a pair of sequences flanking or framing the target sequence to be amplified are chosen to serve as primer sequences, requiring only that the sequences lie on opposite strands of the bacteriophage DNA and that the 3′ ends of each sequence lie within 10 kb or less of one another; [3] Southern hybridization wherein an intact bacteriophage genome or fragments produced by restriction digestion are transferred to a membrane following electrophoresis and hybridized with one or more DNA probes consisting of labeled single or double-stranded DNA oligonucleotides with sequences corresponding to an oligonucleotide of interest, followed by stringency washes; and [4] dot blotting wherein an intact bacteriophage genome is directly applied to a membrane following and hybridized with one or more DNA probes consisting of labeled single or double-stranded DNA oligonucleotides with sequences corresponding to an oligonucleotide of interest, followed by stringency washes.

Thus, for example, by hybridization means, candidate phage genomic DNA, which can be digested with a restriction endonuclease, is exposed to one or more oligonucleotides of interest, labeled with a reporter molecule. Suitable controls are included to enable quantification of signal so that it can be determined whether the phage genome contains all of the oligonucleotides of interest, or complements thereof, if the oligonucleotides of interest or mixtures thereof are combined in the probe solution.

Another embodiment comprises a composition of a bacteriophage whose genome contains two or more sequences drawn from the list comprising Table 2. This composition of bacteriophages comprises a group of lytic bacteriophages whose host range is specific for Salmonella species. The bacteriophage in the composition may contain one copy of sequence drawn from the list in Table 2, or it can contain two or more copies of such sequences. In other embodiments, the phage contains any 3, 4, 5, 6, 7, 8, 9, 10, 11, 12, 13, 14, 15, 16, 17, 18, 19, 20, 21, 22, 23, 24, 25, . . . 283, 284, 285, 286, 287, 288 or 289 oligonucleotides of interest. A preferred embodiment comprises a composition of two or more genetically distinct bacteriophages, each of whose genomes contains two or more sequences drawn from the list comprising Table 2. In this embodiment, the genome of each bacteriophage in the composition contain the same sequences drawn from the list in Table 2 as any other bacteriophage in the composition, or it may differ in one, more than one, or all sequences drawn from the list in Table 2. The genomes of bacteriophages in this composition may contain the same number of sequences drawn from the list in Table 2, or they may contain different numbers of such sequences. The genome of each bacteriophage in the composition may contain one copy of sequence drawn from the list in Table 2, or it can contain two or more copies of such sequences.

The invention does not anticipate that all bacteriophage lytic for Listeria monocytogenes fall within the scope of the compositions where the bacterial genomes contain two or more sequences drawn from the list contained in Table 1. Neither does this invention anticipate that the genomes of all bacteriophage lytic for Salmonella species will contain two or more sequences drawn from the list contained in Table 2.

The invention now will be exemplified by the following non-limiting examples.

### EXAMPLES

### Example 1

### Identification of Oligonucleotide Motifs

The oligonucleotide motifs listed in Table 1 and Table 2 were identified using computational methods available to those skilled in the art. The set of Listeria-specific nucleotide motifs in bacteriophage specific for Listeria monocytogenes shown in Table 1 was obtained through analysis of the sequences of Listeria-specific bacteriophages List-1, List-2, List-3, List-4, List-36, List-38, LMA-34, LMA-57, LMA-94, and LMA-148. The detection and isolation of bacteriophages specific for Listeria monocytogenes is well known in the literature, and obtaining the genomic sequence thereof is likewise obvious to all workers skilled in the art. The set of Salmonella-specific nucleotide motifs in bacteriophage specific for Salmonella species shown in Table 2 was obtained through analysis of the sequences of Salmonella-specific bacteriophages SBA-1781, SDT-15, SHM-125, SHM-135, and SPT-1. The detection and isolation of bacteriophages specific for Salmonella is well known in the literature, and obtaining the genomic sequence thereof is likewise obvious to all workers skilled in the art. For each group of genomic bacteriophage sequences, a set of oligonucleotides was computed such that: [1] each oligonucleotide was 3 nucleotides or longer; [2] each oligonucleotide was as long as possible; [3] any oligonucleotide could hybridize to either strand of the bacteriophage genomic sequence; [4] every oligonucleotide was present in every member of the defining group; [5] no oligonucleotide was present in any member of the other group. Hence, for the Salmonella phage group, the initial set of oligonucleotides was determined that were at least 3 nucleotides long, and would discriminate the Salmonella phage from Listeria phage. The initial analysis identified 2,120 oligonucleotides that would hybridize to the Listeria phage specifically based upon their genomic sequences, but not to the Salmonella phage. In the case of Salmonella phage, a total of 7,878 oligonucleotides were identified that would hybridize to the Salmonella phage specifically based upon the genomic sequences, but not to the Listeria phage.

To refine the analysis further, the initial set of oligonucleotides was compared to a set of 407 phage genomes representing the majority of currently known phage genomes. The phage test data set included free phage genomes that had been identified and sequenced; these sequences were extracted from the NCBI database. In addition, approximately 250 phage genomes were prophage genomes extracted from the sequences of the genomes of their bacterial hosts. These prophage were identified by manual curation of the ends of the prophage based on several criteria including DNA sequence repeats, integrase gene homologies, and insertion sites. The overall data set did not include the sequences of List-1, List-2, List-3, List-4, List-36, List-38, LMA-34, LMA-57, LMA-94, LMA-148, SBA-1781, SDT-15, SHM-125, SHM-135, or SPT-1. The assembled bacteriophage database thus was able to serve as an appropriate control in the analysis of the aforementioned bacteriophage. To perform the analysis, the number of matches of each of the candidate oligonucleotides to each of the phage genomes was recorded. Oligonucleotides from Listeria bacteriophage were accepted only if there were no matches to any other bacteriophage other than bacteriophage specific for Listeria monocytogenes. Similarly, oligonucleotides from Salmonella bacteriophage were accepted only if there were no matches to any other bacteriophage other than bacteriophage specific for Salmonella species.

The foregoing analysis resulted in 44 oligonucleotide motifs specific for bacteriophage that infect Listeria that do not occur in other known phage genomes. Likewise, the analysis resulted in 289 oligonucleotides specific for bacteriophage that infect Salmonella that do not occur in other known phage genomes.

TABLE 1

Oligonucleotide Motifs Specific for Bacteriophage
Infecting Listeria monocytogenes

| | | | | |
|---|---|---|---|---|
| | | | Occurrences | Occurrences |
| | | | in Other | in Other |
| | Oligo- | Occurrences | Listeria- | Non-Listeria- |
| Oligo- | nucleotide | in Phage | specific | specific |
| nucleotide | Motif | Test Data | Bacteriophage | Bacteriophage |
| Motif | Sequence | Set | Genomes | Genomes |

| | | | | |
|---|---|---|---|---|
| 1 | ACGATTAAAAGA | 10 | 5 | 0 |
| | (SEQ ID NO:1) | | | |
| 2 | TCTTTTAATCGT | 10 | 5 | 0 |
| | (SEQ ID NO:2) | | | |
| 3 | AACGATTAAAAGA | 10 | 5 | 0 |
| | (SEQ ID NO:3) | | | |
| 4 | TCTTTTAATCGTT | 10 | 5 | 0 |
| | (SEQ ID NO:4) | | | |
| 5 | AGAAAAGGTGAC | 11 | 3 | 0 |
| | (SEQ ID NO:5) | | | |
| 6 | GTCACCTTTTCT | 11 | 3 | 0 |
| | (SEQ ID NO:6) | | | |
| 7 | ACGTGAATTATC | 10 | 3 | 0 |
| | (SEQ ID NO:7) | | | |
| 8 | ATACTCATGAAC | 10 | 3 | 0 |
| | (SEQ ID NO:8) | | | |
| 9 | GATAATTGACGT | 10 | 3 | 0 |
| | (SEQ ID NO:9) | | | |
| 10 | GTTCATGAGTAT | 10 | 3 | 0 |
| | (SEQ ID NO:10) | | | |
| 11 | AAACGATTAAAAG | 10 | 3 | 0 |
| | (SEQ ID NO:11) | | | |
| 12 | ATGCAAGCCTACC | 10 | 3 | 0 |
| | (SEQ ID NO:12) | | | |
| 13 | CTTTTAATCGTTT | 10 | 3 | 0 |
| | (SEQ ID NO:13) | | | |
| 14 | GGTAGGCTTGCAT | 10 | 3 | 0 |
| | (SEQ ID NO:14) | | | |
| 15 | TAACTTAAAATAA | 10 | 3 | 0 |
| | (SEQ ID NO:15) | | | |
| 16 | TACTTACTGCTAA | 10 | 3 | 0 |
| | (SEQ ID NO:16) | | | |
| 17 | TTAGCAGTAAGTA | 10 | 3 | 0 |
| | (SEQ ID NO:17) | | | |
| 18 | TTATTTTAAGTTA | 10 | 3 | 0 |
| | (SEQ ID NO:18) | | | |
| 19 | AAACGATTAAAAGA | 10 | 3 | 0 |
| | (SEQ ID NO:19) | | | |
| 20 | AAAGAAATGATTGT | 10 | 3 | 0 |
| | (SEQ ID NO:20) | | | |
| 21 | ACAATCATTTCTTT | 10 | 3 | 0 |
| | (SEQ ID NO:21) | | | |
| 22 | AGGTAGGCTTGCAT | 10 | 3 | 0 |
| | (SEQ ID NO:22) | | | |
| 23 | ATGCAAGCTACCT | 10 | 3 | 0 |
| | (SEQ ID NO:23) | | | |
| 24 | GTTATTTTAAGTTA | 10 | 3 | 0 |
| | (SEQ ID NO:24) | | | |
| 25 | TAACTTAAAATAAC | 10 | 3 | 0 |
| | (SEQ ID NO:25) | | | |
| 26 | TCTTTTAATCGTTT | 10 | 3 | 0 |
| | (SEQ ID NO:26) | | | |
| 27 | AAATAAGGGAGT | 11 | 2 | 0 |
| | (SEQ ID NO:27) | | | |
| 28 | ACTCCCTTATTT | 11 | 2 | 0 |
| | (SEQ ID NO:28) | | | |
| 29 | GTAATTTACTTA | 10 | 2 | 0 |
| | (SEQ ID NO:29) | | | |
| 30 | TAAGCAGTAATA | 10 | 2 | 0 |
| | (SEQ ID NO:30) | | | |
| 31 | TAAGTAAATTAC | 10 | 2 | 0 |
| | (SEQ ID NO:31) | | | |
| 32 | TATTACTGCTTA | 10 | 2 | 0 |
| | (SEQ ID NO:32) | | | |
| 33 | TACTCTATACA | 10 | 1 | 0 |
| | (SEQ ID NO:33) | | | |
| 34 | TGTATAGAGTA | 10 | 1 | 0 |
| | (SEQ ID NO:34) | | | |
| 35 | AGAGCTTATTCA | 10 | 1 | 0 |
| | (SEQ ID NO:35) | | | |
| 36 | TGAATAAGCTCT | 10 | 1 | 0 |
| | (SEQ ID NO:36) | | | |
| 37 | AGAGCTTATTCAA | 10 | 1 | 0 |
| | (SEQ ID NO:37) | | | |
| 38 | CTAGAAAAAACAG | 10 | 1 | 0 |
| | (SEQ ID NO:38) | | | |
| 39 | CTGTTTTTTCTAG | 10 | 1 | 0 |
| | (SEQ ID NO:39) | | | |
| 40 | TTGAATAAGCTCT | 10 | 1 | 0 |
| | (SEQ ID NO:40) | | | |
| 41 | AAAAATAAGGGA | 11 | 0 | 0 |
| | (SEQ ID NO:41) | | | |
| 42 | TCCCTTATTTTT | 11 | 0 | 0 |
| | (SEQ ID NO:42) | | | |
| 43 | CCCTTATTTTTA | 10 | 0 | 0 |
| | (SEQ ID NO:43) | | | |
| 44 | TAAAAATAAGGG | 10 | 0 | 0 |
| | (SEQ ID NO:44) | | | |

The phage test data set is defined as the genomic sequences of the Listeria-specific bacteriophages List-1, List-2, List-3, List-4, List-36, List-38, LMA-34, LMA-57, LMA-94, and LMA-148, see WO2005059161. The oligonucleotide motifs may occur more than once in any one bacteriophage genome.

TABLE 2

Oligonucleotide Motifs Specific for Bacteriophage
Infecting Salmonella Species

| | | | | |
|---|---|---|---|---|
| | | | Occurrences | Occurrences |
| | | | in Other | in Other Non- |
| | Oligo- | Occurrences | Salmonella- | Salmonella- |
| Oligo- | nucleotide | in Phage | specific | specific |
| nucleotide | Motif | Test Data | Bacteriophage | Bacteriophage |
| Motif | Sequence | Set | Genomes | Genomes |

| | | | | |
|---|---|---|---|---|
| 1 | CGATAGAGTCA | 11 | 1 | 0 |
| | (SEQ ID NO:45) | | | |
| 2 | TGACTCTATCG | 11 | 1 | 0 |
| | (SEQ ID NO:46) | | | |
| 3 | CCGATAGAGTCA | 8 | 1 | 0 |
| | (SEQ ID NO:47) | | | |
| 4 | TGACTCTATCGG | 8 | 1 | 0 |
| | (SEQ ID NO:48) | | | |
| 5 | GTAGCTGCTGCT | 5 | 3 | 0 |
| | (SEQ ID NO:49) | | | |
| 6 | AGCAGCAGCTAC | 5 | 3 | 0 |
| | (SEQ ID NO:50) | | | |
| 7 | CGATAGAGTCAC | 8 | 0 | 0 |
| | (SEQ ID NO:51) | | | |
| 8 | GTGACTCTATCG | 8 | 0 | 0 |
| | (SEQ ID NO:52) | | | |
| 9 | AAACCCATCAAG | 8 | 0 | 0 |
| | (SEQ ID NO:53) | | | |
| 10 | CTTGATGGGTTT | 8 | 0 | 0 |
| | (SEQ ID NO:54) | | | |
| 11 | GTAGCTGCTGCTG | 5 | 3 | 0 |
| | (SEQ ID NO:55) | | | |
| 12 | CAGCAGCAGCTAC | 5 | 3 | 0 |
| | (SEQ ID NO:56) | | | |
| 13 | GTGACTCTATCGG | 8 | 0 | 0 |
| | (SEQ ID NO:57) | | | |
| 14 | CCGATAGAGTCAC | 8 | 0 | 0 |
| | (SEQ ID NO:58) | | | |
| 15 | CACATCAAAGG | 5 | 2 | 0 |
| | (SEQ ID NO:59) | | | |
| 16 | CTCAGTCCTGA | 5 | 2 | 0 |
| | (SEQ ID NO:60) | | | |
| 17 | CAGGACTGAGT | 5 | 2 | 0 |
| | (SEQ ID NO:61) | | | |
| 18 | ACTCAGTCCTG | 5 | 2 | 0 |
| | (SEQ ID NO:62) | | | |
| 19 | AGGTAATTTCC | 5 | 2 | 0 |
| | (SEQ ID NO:63) | | | |
| 20 | TCAGGACTGAG | 5 | 2 | 0 |
| | (SEQ ID NO:64) | | | |
| 21 | CCTTTGATGTG | 5 | 2 | 0 |
| | (SEQ ID NO:65) | | | |
| 22 | GGAAATTACCT | 5 | 2 | 0 |
| | (SEQ ID NO:66) | | | |
| 23 | GTTTGGCTACCA | 5 | 2 | 0 |
| | (SEQ ID NO:67) | | | |
| 24 | TCAGGACTGAGT | 5 | 2 | 0 |
| | (SEQ ID NO:68) | | | |
| 25 | CAGGACTGAGTT | 5 | 2 | 0 |
| | (SEQ ID NO:69) | | | |
| 26 | AGGTAATTTCCC | 5 | 2 | 0 |
| | (SEQ ID NO:70) | | | |
| 27 | GTCCTTGCTAAA | 5 | 2 | 0 |
| | (SEQ ID NO:71) | | | |
| 28 | TTCACGGGCAAT | 5 | 2 | 0 |
| | (SEQ ID NO:72) | | | |
| 29 | ACTCAGTCCTGA | 5 | 2 | 0 |
| | (SEQ ID NO:73) | | | |
| 30 | ATTGCCCGTGAA | 5 | 2 | 0 |
| | (SEQ ID NO:74) | | | |
| 31 | AACTCAGTCCTG | 5 | 2 | 0 |
| | (SEQ ID NO:75) | | | |
| 32 | GGGAAATTACCT | 5 | 2 | 0 |
| | (SEQ ID NO:76) | | | |
| 33 | TGGTAGCCAAAC | 5 | 2 | 0 |
| | (SEQ ID NO:77) | | | |
| 34 | TTTAGCAAGGAC | 5 | 2 | 0 |
| | (SEQ ID NO:78) | | | |
| 35 | ACCGGAGCTTTC | 7 | 0 | 0 |
| | (SEQ ID NO:79) | | | |
| 36 | GAAAGCTCCGGT | 7 | 0 | 0 |
| | (SEQ ID NO:80) | | | |
| 37 | ATGAAGTATTTCT | 5 | 2 | 0 |
| | (SEQ ID NO:81) | | | |
| 38 | AACTCAGTCCTGA | 5 | 2 | 0 |
| | (SEQ ID NO:82) | | | |
| 39 | CATCTGATACACC | 5 | 2 | 0 |
| | (SEQ ID NO:83) | | | |
| 40 | GGTGTATCAGATG | 5 | 2 | 0 |
| | (SEQ ID NO:84) | | | |
| 41 | AGAAATACTTCAT | 5 | 2 | 0 |
| | (SEQ ID NO:85) | | | |
| 42 | TCAGGACTGAGTT | 5 | 2 | 0 |
| | (SEQ ID NO:86) | | | |
| 43 | GAAAGCTCCGGTA | 7 | 0 | 0 |
| | (SEQ ID NO:87) | | | |
| 44 | TACCGGAGCTTTC | 7 | 0 | 0 |
| | (SEQ ID NO:88) | | | |
| 45 | AGATATGGAAGAAG | 5 | 2 | 0 |
| | (SEQ ID NO:89) | | | |
| 46 | CTTCTTCCATATCT | 5 | 2 | 0 |
| | (SEQ ID NO:90) | | | |
| 47 | TCTGGTTAGCA | 5 | 1 | 0 |
| | (SEQ ID NO:91) | | | |
| 48 | ATGCAGAGAGT | 5 | 1 | 0 |
| | (SEQ ID NO:92) | | | |
| 49 | CCGTGTAATGG | 5 | 1 | 0 |
| | (SEQ ID NO:93) | | | |
| 50 | TGCTAACCAGA | 5 | 1 | 0 |
| | (SEQ ID NO:94) | | | |
| 51 | GGTTATACAGA | 5 | 1 | 0 |
| | (SEQ ID NO:95) | | | |
| 52 | TCTGTATAACC | 5 | 1 | 0 |
| | (SEQ ID NO:96) | | | |
| 53 | ACTCTCTGCAT | 5 | 1 | 0 |
| | (SEQ ID NO:97) | | | |
| 54 | TTAGCTCGCAT | 5 | 1 | 0 |
| | (SEQ ID NO:98) | | | |
| 55 | ATGCGAGCTAA | 5 | 1 | 0 |
| | (SEQ ID NO:99) | | | |
| 56 | CCATTACACGG | 5 | 1 | 0 |
| | (SEQ ID NO:100) | | | |
| 57 | GCATTAAGCATG | 5 | 1 | 0 |
| | (SEQ ID NO:101) | | | |
| 58 | CCATTACACGGC | 5 | 1 | 0 |
| | (SEQ ID NO:102) | | | |
| 59 | TCAAGACCCAGG | 5 | 1 | 0 |
| | (SEQ ID NO:103) | | | |
| 60 | GCCGTGTAATGG | 5 | 1 | 0 |
| | (SEQ ID NO:104) | | | |
| 61 | GAGATATCTTTT | 5 | 1 | 0 |
| | (SEQ ID NO:105) | | | |
| 62 | AAAAGATATCTC | 5 | 1 | 0 |
| | (SEQ ID NO:106) | | | |
| 63 | CCTGGGTCTTGA | 5 | 1 | 0 |
| | (SEQ ID NO:107) | | | |
| 64 | CACATCAAAGGC | 5 | 1 | 0 |
| | (SEQ ID NO:108) | | | |
| 65 | GAGCAGGACTTT | 5 | 1 | 0 |
| | (SEQ ID NO:109) | | | |
| 66 | GCTCTTTGCTTC | 5 | 1 | 0 |
| | (SEQ ID NO:110) | | | |
| 67 | ACTTACACCAAA | 5 | 1 | 0 |
| | (SEQ ID NO:111) | | | |
| 68 | GCGTTTGATGTG | 5 | 1 | 0 |
| | (SEQ ID NO:112) | | | |
| 69 | GAAGCAAAGAGC | 5 | 1 | 0 |
| | (SEQ ID NO:113) | | | |
| 70 | TTTGGTGTAAGT | 5 | 1 | 0 |
| | (SEQ ID NO:114) | | | |
| 71 | ATACTCACCAAA | 5 | 1 | 0 |
| | (SEQ ID NO:115) | | | |
| 72 | CATGCTTAATGC | 5 | 1 | 0 |
| | (SEQ ID NO:116) | | | |
| 73 | AAAGTCCTGGTC | 5 | 1 | 0 |
| | (SEQ ID NO:117) | | | |
| 74 | TCCATTGCAGAA | 5 | 1 | 0 |
| | (SEQ ID NO:118) | | | |
| 75 | TTTGGTGAGTAT | 5 | 1 | 0 |
| | (SEQ ID NO:119) | | | |
| 76 | TCAGAAGTTCTC | 5 | 1 | 0 |
| | (SEQ ID NO:120) | | | |
| 77 | TTCTGCAATGGA | 5 | 1 | 0 |
| | (SEQ ID NO:121) | | | |
| 78 | GAGAACTTCTGA | 5 | 1 | 0 |
| | (SEQ ID NO:122) | | | |
| 79 | GATACAGGTTTAT | 5 | 1 | 0 |
| | (SEQ ID NO:123) | | | |
| 80 | AAGCTTCTTGAAA | 5 | 1 | 0 |
| | (SEQ ID NO:124) | | | |
| 81 | TCAAGACCCAGGC | 5 | 1 | 0 |
| | (SEQ ID NO:125) | | | |
| 82 | TGTCCTTGCTAAA | 5 | 1 | 0 |
| | (SEQ ID NO:126) | | | |
| 83 | AGAAAAGTTGCTG | 5 | 1 | 0 |
| | (SEQ ID NO:127) | | | |
| 84 | GCCTGGGTCTTGA | 5 | 1 | 0 |
| | (SEQ ID NO:128) | | | |
| 85 | AGCAGGACTTTGG | 5 | 1 | 0 |
| | (SEQ ID NO:129) | | | |
| 86 | ATAAACCTGTATC | 5 | 1 | 0 |
| | (SEQ ID NO:130) | | | |
| 87 | TCAAACCCATCAA | 5 | 1 | 0 |
| | (SEQ ID NO:131) | | | |
| 88 | TTTCAAGAAGCTT | 5 | 1 | 0 |
| | (SEQ ID NO:132) | | | |
| 89 | CAAAGTCCTGCTC | 5 | 1 | 0 |
| | (SEQ ID NO:133) | | | |
| 90 | AGATATTTGAAGT | 5 | 1 | 0 |
| | (SEQ ID NO:134) | | | |
| 91 | CAGCAACTTTTCT | 5 | 1 | 0 |
| | (SEQ ID NO:135) | | | |
| 92 | ACTTCAAATATCT | 5 | 1 | 0 |
| | (SEQ ID NO:136) | | | |
| 93 | GAGCAGGACTTTG | 5 | 1 | 0 |
| | (SEQ ID NO:137) | | | |
| 94 | TTTAGCAAGGACA | 5 | 1 | 0 |
| | (SEQ ID NO:138) | | | |
| 95 | CCAAAGTCCTGCT | 5 | 1 | 0 |
| | (SEQ ID NO:139) | | | |
| 96 | GAGATATCTTTTG | 5 | 1 | 0 |
| | (SEQ ID NO:140) | | | |
| 97 | TTGATGGGTTTGA | 5 | 1 | 0 |
| | (SEQ ID NO:141) | | | |
| 98 | CAAAAGATATCTC | 5 | 1 | 0 |
| | (SEQ ID NO:142) | | | |
| 99 | GAGCAGGACTTTGG | 5 | 1 | 0 |
| | (SEQ ID NO:143) | | | |
| 100 | CAGCAACTTTTCTT | 5 | 1 | 0 |
| | (SEQ ID NO:144) | | | |
| 101 | CCAAAGTCCTGCTC | 5 | 1 | 0 |
| | (SEQ ID NO:145) | | | |
| 102 | AAGAAAAGTTGCTG | 5 | 1 | 0 |
| | (SEQ ID NO:146) | | | |
| 103 | TTAACAAAATAACT | 6 | 0 | 0 |
| | (SEQ ID NO:147) | | | |
| 104 | AGTTATTTTGTTAA | 6 | 0 | 0 |
| | (SEQ ID NO:148) | | | |
| 105 | TCTTCTTTTATCTCA | 5 | 1 | 0 |
| | (SEQ ID NO:149) | | | |
| 106 | TGAGATAAAAGAAGA | 5 | 1 | 0 |
| | (SEQ ID NO:150) | | | |
| 107 | TGCAGAGTACC | 5 | 0 | 0 |
| | (SEQ ID NO:151) | | | |
| 108 | ATTGTTTCGTGC | 5 | 0 | 0 |
| | (SEQ ID NO:152) | | | |
| 109 | CTACGCTCCTA | 5 | 0 | 0 |
| | (SEQ ID NO:153) | | | |
| 110 | TAGGTACTCTG | 5 | 0 | 0 |
| | (SEQ ID NO:154) | | | |
| 111 | TAGGAGCGTAG | 5 | 0 | 0 |
| | (SEQ ID NO:155) | | | |
| 112 | AAGTACTATGC | 5 | 0 | 0 |
| | (SEQ ID NO:156) | | | |
| 113 | GGTACTCTGCA | 5 | 0 | 0 |
| | (SEQ ID NO:157) | | | |
| 114 | CAGAGTACCTA | 5 | 0 | 0 |
| | (SEQ ID NO:158) | | | |
| 115 | CTTAGTGCGAG | 5 | 0 | 0 |
| | (SEQ ID NO:159) | | | |
| 116 | CACCTTCTCTA | 5 | 0 | 0 |
| | (SEQ ID NO:160) | | | |
| 117 | GCACGAACAAT | 5 | 0 | 0 |
| | (SEQ ID NO:161) | | | |
| 118 | CACTAACTGAT | 5 | 0 | 0 |
| | (SEQ ID NO:162) | | | |
| 119 | ATCAGTTAGTG | 5 | 0 | 0 |
| | (SEQ ID NO:163) | | | |
| 120 | GTAGGAGCGTA | 5 | 0 | 0 |
| | (SEQ ID NO:164) | | | |
| 121 | CTCGCAGTAAG | 5 | 0 | 0 |
| | (SEQ ID NO:165) | | | |
| 122 | TAGAGAAGGTG | 5 | 0 | 0 |
| | (SEQ ID NO:166) | | | |
| 123 | TACGCTCGTAC | 5 | 0 | 0 |
| | (SEQ ID NO:167) | | | |
| 124 | GCATAGTACTT | 5 | 0 | 0 |
| | (SEQ ID NO:168) | | | |
| 125 | CTTATGACGAAC | 5 | 0 | 0 |
| | (SEQ ID NO:169) | | | |
| 126 | GGCTCATCTGTA | 5 | 0 | 0 |
| | (SEQ ID NO:170) | | | |
| 127 | GAACCAGAAAGT | 5 | 0 | 0 |
| | (SEQ ID NO:171) | | | |
| 128 | ACTTTCTGGTTC | 5 | 0 | 0 |
| | (SEQ ID NO:172) | | | |
| 129 | TATGTTTCAGAA | 5 | 0 | 0 |
| | (SEQ ID NO:173) | | | |
| 130 | GCTGGGACAAGT | 5 | 0 | 0 |
| | (SEQ ID NO:174) | | | |
| 131 | GCATGGCTAACC | 5 | 0 | 0 |
| | (SEQ ID NO:175) | | | |
| 132 | CATGCAGAGAGT | 5 | 0 | 0 |
| | (SEQ ID NO:176) | | | |
| 133 | TGCGTAACCATG | 5 | 0 | 0 |
| | (SEQ ID NO:177) | | | |
| 134 | TTCAGCAAGAAC | 5 | 0 | 0 |
| | (SEQ ID NO:178) | | | |
| 135 | AAGTCTGATAAC | 5 | 0 | 0 |
| | (SEQ ID NO:179) | | | |
| 136 | CTGCCCAGTGAT | 5 | 0 | 0 |
| | (SEQ ID NO:180) | | | |
| 137 | TGGGTGCTCTCT | 5 | 0 | 0 |
| | (SEQ ID NO:181) | | | |
| 138 | GCATAGTACTTG | 5 | 0 | 0 |
| | (SEQ ID NO:182) | | | |
| 139 | TCTCTGCATGTA | 5 | 0 | 0 |
| | (SEQ ID NO:183) | | | |
| 140 | CATGGTTACGCA | 5 | 0 | 0 |
| | (SEQ ID NO:184) | | | |
| 141 | GATTTTACCATT | 5 | 0 | 0 |
| | (SEQ ID NO:185) | | | |
| 142 | GTTCGTCATAAG | 5 | 0 | 0 |
| | (SEQ ID NO:186) | | | |
| 143 | GGTTACGCATCG | 5 | 0 | 0 |
| | (SEQ ID NO:187) | | | |
| 144 | CAGATGCTTTGA | 5 | 0 | 0 |
| | (SEQ ID NO:188) | | | |
| 145 | TGCAGAGTACCT | 5 | 0 | 0 |
| | (SEQ ID NO:189) | | | |
| 146 | TACAGATGAGCC | 5 | 0 | 0 |
| | (SEQ ID NO:190) | | | |
| 147 | AATGGTAAAATC | 5 | 0 | 0 |
| | (SEQ ID NO:191) | | | |
| 148 | CCTTATGACGAA | 5 | 0 | 0 |
| | (SEQ ID NO:192) | | | |
| 149 | GTTCTTGCTGAA | 5 | 0 | 0 |
| | (SEQ ID NO:193) | | | |
| 150 | CAAGTACTATGC | 5 | 0 | 0 |
| | (SEQ ID NO:194) | | | |
| 151 | GTAGGAGCGTAG | 5 | 0 | 0 |
| | (SEQ ID NO:195) | | | |
| 152 | ACAAAGACCTTG | 5 | 0 | 0 |
| | (SEQ ID NO:196) | | | |
| 153 | TTCTGAAACATA | 5 | 0 | 0 |
| | (SEQ ID NO:197) | | | |
| 154 | GCAGTGGTGGAA | 5 | 0 | 0 |
| | (SEQ ID NO:198) | | | |
| 155 | TTCCACCACTGC | 5 | 0 | 0 |
| | (SEQ ID NO:199) | | | |
| 156 | ATCACTGGGCAG | 5 | 0 | 0 |
| | (SEQ ID NO:200) | | | |
| 157 | TTGAAGGCTTTG | 5 | 0 | 0 |
| | (SEQ ID NO:201) | | | |
| 158 | CTCGCAGTAAGG | 5 | 0 | 0 |
| | (SEQ ID NO:202) | | | |
| 159 | ACTCTCTGCATG | 5 | 0 | 0 |
| | (SEQ ID NO:203) | | | |
| 160 | TCTGGAGTTATC | 5 | 0 | 0 |
| | (SEQ ID NO:204) | | | |
| 161 | CTTCGAACGCTT | 5 | 0 | 0 |
| | (SEQ ID NO:205) | | | |
| 162 | TACTCACCAAAT | 5 | 0 | 0 |
| | (SEQ ID NO:206) | | | |
| 163 | CCTTACTGCGAG | 5 | 0 | 0 |
| | (SEQ ID NO:207) | | | |
| 164 | TACATGCAGAGA | 5 | 0 | 0 |
| | (SEQ ID NO:208) | | | |
| 165 | AGATGGCAGAAA | 5 | 0 | 0 |
| | (SEQ ID NO:209) | | | |
| 166 | ATTGCAGAGAGT | 5 | 0 | 0 |
| | (SEQ ID NO:210) | | | |
| 167 | CAAGGTCTTTGT | 5 | 0 | 0 |
| | (SEQ ID NO:211) | | | |
| 168 | CAAAGCCTTCAA | 5 | 0 | 0 |
| | (SEQ ID NO:212) | | | |
| 169 | GTTATCAGACTT | 5 | 0 | 0 |
| | (SEQ ID NO:213) | | | |
| 170 | ATCAAGCGCAGT | 5 | 0 | 0 |
| | (SEQ ID NO:214) | | | |
| 171 | TTTGAGCATTAC | 5 | 0 | 0 |
| | (SEQ ID NO:215) | | | |
| 172 | GTAATGCTCAAA | 5 | 0 | 0 |
| | (SEQ ID NO:216) | | | |
| 173 | AAGCGTTCGAAG | 5 | 0 | 0 |
| | (SEQ ID NO:217) | | | |
| 174 | AGAGAGCACCCA | 5 | 0 | 0 |
| | (SEQ ID NO:218) | | | |
| 175 | GATAACTCCAGA | 5 | 0 | 0 |
| | (SEQ ID NO:219) | | | |
| 176 | AGTCTCTGCAAT | 5 | 0 | 0 |
| | (SEQ ID NO:220) | | | |
| 177 | TTCGTCATAAGG | 5 | 0 | 0 |
| | (SEQ ID NO:221) | | | |
| 178 | CGATGCGTAACC | 5 | 0 | 0 |
| | (SEQ ID NO:222) | | | |
| 179 | AGGTACTCTGCA | 5 | 0 | 0 |
| | (SEQ ID NO:223) | | | |
| 180 | TTTCTGCCATCT | 5 | 0 | 0 |
| | (SEQ ID NO:224) | | | |
| 181 | ATTTGGTGAGTA | 5 | 0 | 0 |
| | (SEQ ID NO:225) | | | |
| 182 | CTACGCTCCTAC | 5 | 0 | 0 |
| | (SEQ ID NO:226) | | | |
| 183 | GGTTAGCCATGC | 5 | 0 | 0 |
| | (SEQ ID NO:227) | | | |
| 184 | ACTGCGCTTGAT | 5 | 0 | 0 |
| | (SEQ ID NO:228) | | | |
| 185 | CAAAGCATCTGG | 5 | 0 | 0 |
| | (SEQ ID NO:229) | | | |
| 186 | TCAAAGCATCTG | 5 | 0 | 0 |
| | (SEQ ID NO:230) | | | |
| 187 | CCAGATGCTTTG | 5 | 0 | 0 |
| | (SEQ ID NO:231) | | | |
| 188 | ACTTGTCCCAGC | 5 | 0 | 0 |
| | (SEQ ID NO:232) | | | |
| 189 | GGTGGTATCTTTG | 5 | 0 | 0 |
| | (SEQ ID NO:233) | | | |
| 190 | ACTTCTCTTGCGA | 5 | 0 | 0 |
| | (SEQ ID NO:234) | | | |
| 191 | ACCAAAGATACCA | 5 | 0 | 0 |
| | (SEQ ID NO:235) | | | |
| 192 | CCAGATGCTTTGA | 5 | 0 | 0 |
| | (SEQ ID NO:236) | | | |
| 193 | TGTTCTTGCTGAA | 5 | 0 | 0 |
| | (SEQ ID NO:237) | | | |
| 194 | ACTGATGGAAAAC | 5 | 0 | 0 |
| | (SEQ ID NO:238) | | | |
| 195 | AATCTATCAGCAA | 5 | 0 | 0 |
| | (SEQ ID NO:239) | | | |
| 196 | AATCACTGGGCAG | 5 | 0 | 0 |
| | (SEQ ID NO:240) | | | |
| 197 | CTTAGCAATCTGT | 5 | 0 | 0 |
| | (SEQ ID NO:241) | | | |
| 198 | AAAGGAGACTTAA | 5 | 0 | 0 |
| | (SEQ ID NO:242) | | | |
| 199 | CATTCTCTGATTT | 5 | 0 | 0 |
| | (SEQ ID NO:243) | | | |
| 200 | TTTACATCTCTTC | 5 | 0 | 0 |
| | (SEQ ID NO:244) | | | |
| 201 | TGTTGAGTATTTC | 5 | 0 | 0 |
| | (SEQ ID NO:245) | | | |
| 202 | CAGATGCTTTGAT | 5 | 0 | 0 |
| | (SEQ ID NO:246) | | | |
| 203 | ATACTCACCAAAT | 5 | 0 | 0 |
| | (SEQ ID NO:247) | | | |
| 204 | CTTAACTCCTGAA | 5 | 0 | 0 |
| | (SEQ ID NO:248) | | | |
| 205 | TGGTATCTTTGGT | 5 | 0 | 0 |
| | (SEQ ID NO:249) | | | |
| 206 | TATTTTAATTGAG | 5 | 0 | 0 |
| | (SEQ ID NO:250) | | | |
| 207 | TTCAGCAAGAACA | 5 | 0 | 0 |
| | (SEQ ID NO:251) | | | |
| 208 | TTGCTTCAAAGTT | 5 | 0 | 0 |
| | (SEQ ID NO:252) | | | |
| 209 | GTTTTCCATCAGT | 5 | 0 | 0 |
| | (SEQ ID NO:253) | | | |
| 210 | TGCTGTGGTTGTT | 5 | 0 | 0 |
| | (SEQ ID NO:254) | | | |
| 211 | TACATGCAGAGAG | 5 | 0 | 0 |
| | (SEQ ID NO:255) | | | |
| 212 | CAAACCCATCAAG | 5 | 0 | 0 |
| | (SEQ ID NO:256) | | | |
| 213 | ATTTGGTGAGTAT | 5 | 0 | 0 |
| | (SEQ ID NO:257) | | | |
| 214 | AAACCCATCAAGT | 5 | 0 | 0 |
| | (SEQ ID NO:258) | | | |
| 215 | CTCAATTAAAATA | 5 | 0 | 0 |
| | (SEQ ID NO:259) | | | |
| 216 | CTTGATGGGTTTG | 5 | 0 | 0 |
| | (SEQ ID NO:260) | | | |
| 217 | AAATCAGAGAATG | 5 | 0 | 0 |
| | (SEQ ID NO:261) | | | |
| 218 | AACGTTAATATTC | 5 | 0 | 0 |
| | (SEQ ID NO:262) | | | |
| 219 | AACAACCACAGCA | 5 | 0 | 0 |
| | (SEQ ID NO:263) | | | |
| 220 | TTAGAGCTTTACG | 5 | 0 | 0 |
| | (SEQ ID NO:264) | | | |
| 221 | ACAGATTGCTAAG | 5 | 0 | 0 |
| | (SEQ ID NO:265) | | | |
| 222 | ATCAAAGCATCTG | 5 | 0 | 0 |
| | (SEQ ID NO:266) | | | |
| 223 | CAAAGATACCACC | 5 | 0 | 0 |
| | (SEQ ID NO:267) | | | |
| 224 | TTAAGTCTCCTTT | 5 | 0 | 0 |
| | (SEQ ID NO:268) | | | |
| 225 | ACTTGATGGGTTT | 5 | 0 | 0 |
| | (SEQ ID NO:269) | | | |
| 226 | CAATTAACTCACC | 5 | 0 | 0 |
| | (SEQ ID NO:270) | | | |
| 227 | TCAAAGCATCTGG | 5 | 0 | 0 |
| | (SEQ ID NO:271) | | | |
| 228 | GATTTTACCATTA | 5 | 0 | 0 |
| | (SEQ ID NO:272) | | | |
| 229 | GAAGAGATGTAAA | 5 | 0 | 0 |
| | (SEQ ID NO:273) | | | |
| 230 | AGAACCAGAAAGT | 5 | 0 | 0 |
| | (SEQ ID NO:274) | | | |
| 231 | GAATATTAACGTT | 5 | 0 | 0 |
| | (SEQ ID NO:275) | | | |
| 232 | ACATGCAGAGAGT | 5 | 0 | 0 |
| | (SEQ ID NO:276) | | | |
| 233 | GTTCGTCATAAGG | 5 | 0 | 0 |
| | (SEQ ID NO:277) | | | |
| 234 | AACTTTGAAGCAA | 5 | 0 | 0 |
| | (SEQ ID NO:278) | | | |
| 235 | TCGCAAGAGAAGT | 5 | 0 | 0 |
| | (SEQ ID NO:279) | | | |
| 236 | TTCAGGAGTTAAG | 5 | 0 | 0 |
| | (SEQ ID NO:280) | | | |
| 237 | CTGCCCAGTGATT | 5 | 0 | 0 |
| | (SEQ ID NO:281) | | | |
| 238 | ACTCTCTGCATGT | 5 | 0 | 0 |
| | (SEQ ID NO:282) | | | |
| 239 | CCTTATGACGAAC | 5 | 0 | 0 |
| | (SEQ ID NO:283) | | | |
| 240 | TTGCTGATAGATT | 5 | 0 | 0 |
| | (SEQ ID NO:284) | | | |
| 241 | AATGGTAAAATCT | 5 | 0 | 0 |
| | (SEQ ID NO:285) | | | |
| 242 | GGTGAGTTAATTG | 5 | 0 | 0 |
| | (SEQ ID NO:286) | | | |
| 243 | AGATTTTACCATT | 5 | 0 | 0 |
| | (SEQ ID NO:287) | | | |
| 244 | ACTTTCTGGTTCT | 5 | 0 | 0 |
| | (SEQ ID NO:288) | | | |
| 245 | GAAATACTCAACA | 5 | 0 | 0 |
| | (SEQ ID NO:289) | | | |
| 246 | CGTAAAGCTCTAA | 5 | 0 | 0 |
| | (SEQ ID NO:290) | | | |
| 247 | GTCTCTGCATGTA | 5 | 0 | 0 |
| | (SEQ ID NO:291) | | | |
| 248 | TAATGGTAAAATC | 5 | 0 | 0 |
| | (SEQ ID NO:292) | | | |
| 249 | CCAAAGATACCACC | 5 | 0 | 0 |
| | (SEQ ID NO:293) | | | |
| 250 | AAACCCATCAAGTA | 5 | 0 | 0 |
| | (SEQ ID NO:294) | | | |
| 251 | GGTGGTATCTTTGG | 5 | 0 | 0 |
| | (SEQ ID NO:295) | | | |
| 252 | GTGGTATCTTTGGT | 5 | 0 | 0 |
| | (SEQ ID NO:296) | | | |
| 253 | ACTTTCTGGTTCTC | 5 | 0 | 0 |
| | (SEQ ID NO:297) | | | |
| 254 | ACTTCTCTTGCGAT | 5 | 0 | 0 |
| | (SEQ ID NO:298) | | | |
| 255 | ACCAAAGATACCAC | 5 | 0 | 0 |
| | (SEQ ID NO:299) | | | |
| 256 | CTTGATGGGTTTGA | 5 | 0 | 0 |
| | (SEQ ID NO:300) | | | |
| 257 | AACGTTAATATTCA | 5 | 0 | 0 |
| | (SEQ ID NO:301) | | | |
| 258 | ATCAAAGCATCTGG | 5 | 0 | 0 |
| | (SEQ ID NO:302) | | | |
| 259 | AGATTTTACCATTA | 5 | 0 | 0 |
| | (SEQ ID NO:303) | | | |
| 260 | TCAAACCCATCAAG | 5 | 0 | 0 |
| | (SEQ ID NO:304) | | | |
| 261 | CTAATGGTAAAATC | 5 | 0 | 0 |
| | (SEQ ID NO:305) | | | |
| 262 | CAATTAACTCACCA | 5 | 0 | 0 |
| | (SEQ ID NO:306) | | | |
| 263 | ATCGCAAGAGAAGT | 5 | 0 | 0 |
| | (SEQ ID NO:307) | | | |
| 264 | TGTTGAGTATTTCT | 5 | 0 | 0 |
| | (SEQ ID NO:308) | | | |
| 265 | CCAGATGCTTTGAT | 5 | 0 | 0 |
| | (SEQ ID NO:309) | | | |
| 266 | GAGAACCAGAAAGT | 5 | 0 | 0 |
| | (SEQ ID NO:310) | | | |
| 267 | TACTTGATGGGTTT | 5 | 0 | 0 |
| | (SEQ ID NO:311) | | | |
| 268 | ACTCTCTGCATGTA | 5 | 0 | 0 |
| | (SEQ ID NO:312) | | | |
| 269 | GATTTTACCATTAG | 5 | 0 | 0 |
| | (SEQ ID NO:313) | | | |
| 270 | TGAATATTAACGTT | 5 | 0 | 0 |
| | (SEQ ID NO:314) | | | |
| 271 | TACATGCAGAGAGT | 5 | 0 | 0 |
| | (SEQ ID NO:315) | | | |
| 272 | ACTTAGCAATCTGT | 5 | 0 | 0 |
| | (SEQ ID NO:316) | | | |
| 273 | ACAGATTGCTAAGT | 5 | 0 | 0 |
| | (SEQ ID NO:317) | | | |
| 274 | TAATGGTAAAATCT | 5 | 0 | 0 |
| | (SEQ ID NO:318) | | | |
| 275 | TGGTGAGTTAATTG | 5 | 0 | 0 |
| | (SEQ ID NO:319) | | | |
| 276 | GAATTGCAGAAAGC | 5 | 0 | 0 |
| | (SEQ ID NO:320) | | | |
| 277 | GCTTTCTGCAATTC | 5 | 0 | 0 |
| | (SEQ ID NO:321) | | | |
| 278 | TGAATTGCAGAAAGC | 5 | 0 | 0 |
| | (SEQ ID NO:322) | | | |
| 279 | TAATGGTAAAATCT | 5 | 0 | 0 |
| | (SEQ ID NO:323) | | | |
| 280 | ACTTCTCTTGCGATT | 5 | 0 | 0 |
| | (SEQ ID NO:324) | | | |
| 281 | AATCGCAAGAGAAGT | 5 | 0 | 0 |
| | (SEQ ID NO:325) | | | |
| 282 | TGTTGAGTATTTCTT | 5 | 0 | 0 |
| | (SEQ ID NO:326) | | | |
| 283 | GCTTTCTGCAATTCA | 5 | 0 | 0 |
| | (SEQ ID NO:327) | | | |
| 284 | AAGAAATACTCAACA | 5 | 0 | 0 |
| | (SEQ ID NO:328) | | | |
| 285 | AGATTTTACCATTAG | 5 | 0 | 0 |
| | (SEQ ID NO:329) | | | |
| 286 | GGTGGTATCTTTGGT | 5 | 0 | 0 |
| | (SEQ ID NO:330) | | | |
| 287 | ACCAAAGATACCACC | 5 | 0 | 0 |
| | (SEQ ID NO:331) | | | |
| 288 | TTACTTGATGGGTTT | 5 | 0 | 0 |
| | (SEQ ID NO:332) | | | |
| 289 | AAACCCATCAAGTAA | 5 | 0 | 0 |
| | (SEQ ID NO:333) | | | |

The phage test data set is defined as the genomic sequences of the Salmonella-specific bacteriophages SBA-1781, SDT-15, SHM-125, SHM-135, and SPT-1, see WO2005027829. The oligonucleotide motifs may occur more than once in any bacteriophage genomic sequence.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

All references cited herein are herein incorporated by reference in entirety.
1. Salgado, C. J., Zayas, M., and Villafane, R., Homology between two different Salmonella phages: Salmonella enterica serovar Typhimurium phage P22 and Salmonella enterica serovar Anatum var. 15+phageepsilon34, Virus Genes, 29, 87 (2004).
2. Tetart, F., Desplats, C., and Krisch, H. M., Genome plasticity in the distal tail fiber locus of the T-even bacteriophage: recombination between conserved motifs swaps adhesin specificity, Journal of Molecular Biology, 282, 543 (1998).
3. Marciel, A. M., Kapur, V., and Musser, J. M., Molecular population genetic analysis of a Streptococcus pyogenes bacteriophage-encoded hyaluronidase gene: recombination contributes to allelic variation, Microbial Pathogenesis, 22, 209 (1997).
4. Loessner, M. J., Kramer, K., Ebel, F., and Scherer, S., C-terminal domains of Listeria monocytogenes bacteriophage murein hydrolases determine specific recognition and high-affinity binding to bacterial cell wall carbohydrates, Molecular Microbiology, 44, 335 (2002).
5. Chua, J. E., Manning, P. A., and Morona, R., The Shigella flexneri bacteriophage Sf6 tailspike protein (TSP)/endorhamnosidase is related to the bacteriophage P22 TSP and has a motif common to exo- and endoglycanases, and C-5 epimerases, Microbiology, 145, 1649 (1999).
6. Chipman, P. R., Agbandje-McKenna, M., Renaudin, J., Baker, T. S., and McKenna, R., Structural analysis of the Spiroplasma virus, SpV4: implications for evolutionary variation to obtain host diversity among the Microviridae, Structure, 6, 135 (1998).
7. Dorgai, L., Sloan, S., and Weisberg, R. A., Recognition of core binding sites by bacteriophage integrases, Journal of Molecular Biology, 277, 1059 (1998).
8. Kaneko, J., Kimura, T., Narita, S., Tomita, T., and Kamio, Y., Complete nucleotide sequence and molecular characterization of the temperate staphylococcal bacteriophage phiPVL carrying Panton-Valentine leukocidin genes, Gene, 215, 57 (1998).
9. Salmi, D., Magrini, V., Hartzell, P. L., and Youderian, P., Genetic determinants of immunity and integration of temperate Myxococcus xanthus phage Mx8, Journal of Bacteriology, 180, 614 (1998).
10. Crutz-Le Coq, A. M., Cesselin, B., Commissaire, J., and Anba, J., Sequence analysis of the lactococcal bacteriophage bIL170: insights into structural proteins and HNH endonucleases in dairy phages, Microbiology, 148, 985 (2002).
11. Gottlieb, P., Potgieter, C., Wei, H., and Toporovsky, I., Characterization of phi12, a bacteriophage related to phi6: nucleotide sequence of the large double-stranded RNA, Virology, 295, 266 (2002).
12. Gottlieb, P., Wei, H., Potgieter, C., and Toporovsky, I., Characterization of phi 12, a bacteriophage related to phi 6: nucleotide sequence of the small and middle double-stranded RNA, Virology, 293, 118 (2002).
13. Tu, A. H., Voelker, L. L., Shen, X., and Dybvig, K., Complete nucleotide sequence of the mycoplasma virus P1 genome, Plasmid, 45, 122 (2001).
14. Weisberg, R. A., Gottesmann, M. E., Hendrix, R. W., and Little, J. W., Family values in the age of genomics: comparative analyses of temperate bacteriophage HK022, Annual Review of Genetics; 33, 565 (1999).
15. Pfister, P., Wasserfallen, A., Stettler, R., and Leisinger, T., Molecular analysis of Methanobacterium phage psiM2, Molecular Microbiology, 30, 233 (1998).
16. Mesyanzhinov, V. V., Robben, J., Grymonprez, B., Kostyuchenko, V. A., Bourkaltseva, M. V., Sykilinda, N. N., Krylov, V. N., and Volckaert, G., The genome of bacteriophage phiKZ of Pseudomonas aeruginosa, Journal of Molecular Biology, 317, 1 (2002).
17. Altieri, A. S., Mazzulla, M. J., Zhou, H., Costantino, N., Court, D. L., and Byrd, R. A., Sequential assignments and secondary structure of the RNA-binding transcriptional regulator NusB, FEBS Letters, 415, 221 (1997).
18. Verheust, C., Fornelos, N., and Mahillon, J., GIL16, a new gram-positive tectiviral phage related to the Bacillus thuringiensis GIL01 and the Bacillus cereus pBClin15 elements, Journal of Bacteriology, 187, 1966 (2005).
19. Akulenko, N. V., Ivashina, T. V., Shaloiko, L. A., Shliapnikov, M. G., and Ksenzenko, V. N., [Novel site-specific endonucleases F-TflI, F-TflII and F-TflIV encoded by the bacteriophage T5], Molekuliarnaia Biologiia, 38, 632 (2004).
20. Cannistraro, V. J., and Taylor, J. S., DNA-thumb interactions and processivity of T7 DNA polymerase in comparison to yeast polymerase eta, Journal of Biological Chemistry, 279, 18288 (2004).
21. Lebars, I., Hu, R. M., Lallemand, J. Y., Uzan, M., and Bontems, F., Role of the substrate conformation and of the S1 protein in the cleavage efficiency of the T4 endoribonuclease RegB, Journal of Biological Chemistry, 276, 13264 (2001).
22. Sanson, B., Hu, R. M., Troitskayadagger, E., Mathy, N., and Uzan, M., Endoribonuclease RegB from bacteriophage T4 is necessary for the degradation of early but not middle or late mRNAs, Journal of Molecular Biology, 297, 1063 (2000).
23. Lee, S. J., and Richardson, C. C., Essential lysine residues in the RNA polymerase domain of the gene 4 primase-helicase of bacteriophage T7, Journal of Biological Chemistry, 276, 49419 (2001).
24. Benevides, J. M., Bondre, P., Duda, R. L., Hendrix, R. W., and Thomas, G. J., Jr., Domain structures and roles in bacteriophage HK97 capsid assembly and maturation, Biochemistry, 43, 5428 (2004).
25. Pederson, D. M., Welsh, L. C., Marvin, D. A., Sampson, M., Perham, R. N., Yu, M., and Slater, M. R., The protein capsid of filamentous bacteriophage PH75 from Thermus thermophilus, Journal of Molecular Biology, 309, 401 (2001).
26. Kim, H. C., and Gottesman, M. E., Transcription termination by phage HK022 Nun is facilitated by COOH-terminal lysine residues, Journal of Biological Chemistry, 279, 13412 (2004).
27. Kumaraswami, M., Howe, M. M., and Park, H. W., Crystal structure of the Mor protein of bacteriophage Mu, a member of the Mor/C family of transcription activators, Journal of Biological Chemistry, 279, 16581 (2004).
28. Li, N., Zhang, W., White, S. W., and Kriwacki, R. W., Solution structure of the transcriptional activation domain of the bacteriophage T4 protein, MotA, Biochemistry, 40, 4293 (2001).
29. Lindqvist, M., Sandstrom, K., Liepins, V., Stromberg, R., and Graslund, A., Specific metal-ion binding sites in a model of the P4-P6 triple-helical domain of a group I intron, Rna-A Publication of the Rna Society, 7, 1115 (2001).
30. Rutkai, E., Dorgai, L., Sirot, R., Yagil, E., and Weisberg, R. A., Analysis of insertion into secondary attachment sites by phage lambda and by int mutants with altered recombination specificity, Journal of Molecular Biology, 329, 983 (2003).
31. Defenbaugh, D. A., and Nakai, H., A context-dependent ClpX recognition determinant located at the C terminus of phage Mu repressor, Journal of Biological Chemistry, 278, 52333 (2003).
32. Stuart, A. C., Gottesman, M. E., and Palmer, A. G., 3rd, The N-terminus is unstructured, but not dynamically disordered, in the complex between HK022 Nun protein and lambda-phage BoxB RNA hairpin, FEBS Letters, 553, 95 (2003).
33. Mitchell, M. S., and Rao, V. B., Novel and deviant Walker A ATP-binding motifs in bacteriophage large terminase-DNA packaging proteins, Virology, 321, 217 (2004).
34. Benevides, J. M., Juuti, J. T., Tuma, R., Bamford, D. H., and Thomas, G. J., Jr., Characterization of subunit-specific interactions in a double-stranded RNA virus: Raman difference spectroscopy of the phi6 procapsid, Biochemistry, 41, 11946 (2002).
35. Brunel, C., Marquet, R., Romby, P., and Ehresmann, C., RNA loop-loop interactions as dynamic functional motifs, Biochimie, 84, 925 (2002).
36. Mitchell, M. S., Matsuzaki, S., Imai, S., and Rao, V. B., Sequence analysis of bacteriophage T4 DNA packaging/terminase genes 16 and 17 reveals a common ATPase center in the large subunit of viral terminases, Nucleic Acids Research, 30, 4009 (2002).
37. Rao, V. B., and Mitchell, M. S., The N-terminal ATPase site in the large terminase protein gp17 is critically required for DNA packaging in bacteriophage T4, Journal of Molecular Biology, 314, 401 (2001).
38. Rodriguez-Casado, A., Moore, S. D., Prevelige, P. E., Jr., and Thomas, G. J., Jr., Structure of bacteriophage P22 portal protein in relation to assembly: investigation by Raman spectroscopy, Biochemistry, 40, 13583 (2001).
39. Kuebler, D., and Rao, V. B., Functional analysis of the DNA-packaging/terminase protein gp17 from bacteriophage T4, Journal of Molecular Biology, 281, 803 (1998).
40. Parker, M. H., and Prevelige, P. E., Jr., Electrostatic interactions drive scaffolding/coat protein binding and procapsid maturation in bacteriophage P22, Virology, 250, 337 (1998).
41. Tuma, R., Parker, M. H., Weigele, P., Sampson, L., Sun, Y., Krishna, N. R., Casjens, S., Thomas, G. J., Jr., and Prevelige, P. E., Jr., A helical coat protein recognition domain of the bacteriophage P22 scaffolding protein, Journal of Molecular Biology, 281, 81 (1998).
42. Tao, Y., Strelkov, S. V., Mesyanzhinov, V. V., and Rossmann, M. G., Structure of bacteriophage T4 fibritin: a segmented coiled coil and the role of the C-terminal domain, Structure, 5, 789 (1997).
43. White, D. J., Reiter, N. J., Sikkink, R. A., Yu, L., and Rusnak, F., Identification of the high affinity Mn2+ binding site of bacteriophage lambda phosphoprotein phosphatase: effects of metal ligand mutations on electron paramagnetic resonance spectra and phosphatase activities, Biochemistry, 40, 8918 (2001).
44. Eisenbrandt, R., Lazaro, J. M., Salas, M., and de Vega, M., Phi29 DNA polymerase residues Tyr59, His61 and Phe69 of the highly conserved ExoII motif are essential for interaction with the terminal protein, Nucleic Acids Research, 30, 1379 (2002).
45. Galburt, E. A., Pelletier, J., Wilson, G., and Stoddard, B. L., Structure of a tRNA repair enzyme and molecular biology workhorse: T4 polynucleotide kinase.[see comment], Structure, 10, 1249 (2002).
46. Imburgio, D., Anikin, M., and McAllister, W. T., Effects of substitutions in a conserved DX(2)GR sequence motif, found in many DNA-dependent nucleotide polymerases, on transcription by T7 RNA polymerase, Journal of Molecular Biology, 319, 37 (2002).
47. Karpel, R. L., LAST motifs and SMART domains in gene 32 protein: an unfolding story of autoregulation? IUBMB Life, 53, 161 (2002).
48. Lee, S. J., and Richardson, C. C., Interaction of adjacent primase domains within the hexameric gene 4 helicase-primase of bacteriophage T7, Proceedings of the National Academy of Sciences of the United States of America, 99, 12703 (2002).
49. Petrov, V. M., Ng, S. S., and Karam, J. D., Protein determinants of RNA binding by DNA polymerase of the T4-related bacteriophage RB69, Journal of Biological Chemistry, 277, 33041 (2002).
50. Rezende, L. F., Hollis, T., Ellenberger, T., and Richardson, C. C., Essential amino acid residues in the single-stranded DNA-binding protein of bacteriophage T7. Identification of the dimer interface, Journal of Biological Chemistry, 277, 50643 (2002).
51. Sam, M. D., Papagiannis, C. V., Connolly, K. M., Corselli, L., Iwahara, J., Lee, J., Phillips, M., Wojciak, J. M., Johnson, R. C., and Clubb, R. T., Regulation of directionality in bacteriophage lambda site-specific recombination: structure of the Xis protein, Journal of Molecular Biology, 324, 791 (2002).
52. Wojciak, J. M., Sarkar, D., Landy, A., and Clubb, R. T., Arm-site binding by lambda-integrase: solution structure and functional characterization of its amino-terminal domain, Proceedings of the National Academy of Sciences of the United States of America, 99, 3434 (2002).
53. Yeo, H. J., Ziegelin, G., Korolev, S., Calendar, R., Lanka, E., and Waksman, G., Phage P4 origin-binding domain structure reveals a mechanism for regulation of DNA-binding activity by homo- and heterodimerization of winged helix proteins, Molecular Microbiology, 43, 855 (2002).
54. Bravo, A., Serrano-Heras, G., and Salas, M., A single amino acid substitution within a coiled-coil motif changes the assembly of a 53-amino acid protein from two-dimensional sheets to filamentous structures, Journal of Biological Chemistry, 276, 21250 (2001).
55. Moyer, K. E., Kimsey, H. H., and Waldor, M. K., Evidence for a rolling-circle mechanism of phage DNA synthesis from both replicative and integrated forms of CTXphi, Molecular Microbiology, 41, 311 (2001).
56. Radlinska, M., and Bujnicki, J. M., Cloning of enterohemorrhagic Escherichia coli phage VT-2 dam methyltransferase, Acta Microbiologica Polonica, 50, 161 (2001).
57. Schneider, T. D., Strong minor groove base conservation in sequence logos implies DNA distortion or base flipping during replication and transcription initiation, Nucleic Acids Research, 29, 4881 (2001).
58. Valentine, A. M., Ishmael, F. T., Shier, V. K., and Benkovic, S. J., A zinc ribbon protein in DNA replication: primer synthesis and macromolecular interactions by the bacteriophage T4 primase, Biochemistry, 40, 15074 (2001).
59. de Vega, M., Lazaro, J. M., and Salas, M., Phage phi 29 DNA polymerase residues involved in the proper stabilisation of the primer-terminus at the 3′-5′ exonuclease active site, Journal of Molecular Biology, 304, 1 (2000).
60. Hoogstraten, D., Qiao, X., Sun, Y., Hu, A., Onodera, S., and Mindich, L., Characterization of phi8, a bacteriophage containing three double-stranded RNA genomic segments and distantly related to Phi6, Virology, 272, 218 (2000).
61. Makeyev, E. V., and Bamford, D. H., Replicase activity of purified recombinant protein P2 of double-stranded RNA bacteriophage phi6, EMBO Journal, 19, 124 (2000).
62. Moscoso, M., and Suarez, J. E., Characterization of the DNA replication module of bacteriophage A2 and use of its origin of replication as a defense against infection during milk fermentation by Lactobacillus casei, Virology, 273, 101 (2000).
63. Tseng, T. Y., Frick, D. N., and Richardson, C. C., Characterization of a novel DNA primase from the Salmonella typhimurium bacteriophage SP6, Biochemistry, 39, 1643 (2000).
64. Illana, B., Zaballos, A., Blanco, L., and Salas, M., The RGD sequence in phage phi29 terminal protein is required for interaction with phi29 DNA polymerase, Virology, 248, 12 (1998).
65. Foley, S., Bruttin, A., and Brussow, H., Widespread distribution of a group I intron and its three deletion derivatives in the lysin gene of Streptococcus thermophilus bacteriophages, Journal of Virology, 74, 611 (2000).
66. Lucchini, S., Desiere, F., and Brussow, H., Similarly organized lysogeny modules in temperate Siphoviridae from low GC content gram-positive bacteria, Virology, 263, 427 (1999).
67. Desiere, F., Lucchini, S., Bruttin, A., Zwahlen, M. C., and Brussow, H., A highly conserved DNA replication module from Streptococcus thermophilus phages is similar in sequence and topology to a module from Lactococcus lactis phages, Virology, 234, 372 (1997).
68. Sengupta, T. K., Gordon, J., and Spicer, E. K., RegA proteins from phage T4 and RB69 have conserved helix-loop groove RNA binding motifs but different RNA binding specificities, Nucleic Acids Research, 29, 1175 (2001).
69. Nechaev, S., Kamali-Moghaddam, M., Andre, E., Leonetti, J. P., and Geiduschek, E. P., The bacteriophage T4 late-transcription coactivator gp33 binds the flap domain of Escherichia coli RNA polymerase, Proceedings of the National Academy of Sciences of the United States of America, 101, 17365 (2004).
70. Orsini, G., Igonet, S., Pene, C., Sclavi, B., Buckle, M., Uzan, M., and Kolb, A., Phage T4 early promoters are resistant to inhibition by the anti-sigma factor AsiA, Molecular Microbiology, 52, 1013 (2004).
71. Piesiniene, L., Truncaite, L., Zajanckauskaite, A., and Nivinskas, R., The sequences and activities of RegB endoribonucleases of T4-related bacteriophages, Nucleic Acids Research, 32, 5582 (2004).
72. Vieu, E., and Rahmouni, A. R., Dual role of boxB RNA motif in the mechanisms of termination/antitermination at the lambda tR1 terminator revealed in vivo, Journal of Molecular Biology, 339, 1077 (2004).
73. Christie, G. E., Anders, D. L., McAlister, V., Goodwin, T. S., Julien, B., and Calendar, R., Identification of upstream sequences essential for activation of a bacteriophage P2 late promoter, Journal of Bacteriology, 185, 4609 (2003).
74. Cilley, C. D., and Williamson, J. R., Structural mimicry in the phage phi21 N peptide-boxB RNA complex, Rna-A Publication of the Rna Society, 9, 663 (2003).
75. Fromknecht, K., Vogel, P. D., and Wise, J. G., Combinatorial redesign of the DNA binding specificity of a prokaryotic helix-turn-helix repressor, Journal of Bacteriology, 185, 475 (2003).
76. Kim, H. C., Zhou, J. G., Wilson, H. R., Mogilnitskiy, G., Court, D. L., and Gottesman, M. E., Phage HK022 Nun protein represses translation of phage lambda N (transcription termination/translation repression), Proceedings of the National Academy of Sciences of the United States of America, 100, 5308 (2003).
77. Marshall-Batty, K. R., and Nakai, H., Trans-targeting of the phage Mu repressor is promoted by conformational changes that expose its ClpX recognition determinant, Journal of Biological Chemistry, 278, 1612 (2003).
78. Mukhopadhyay, B., Marshall-Batty, K. R., Kim, B. D., O'Handley, D., and Nakai, H., Modulation of phage Mu repressor DNA binding and degradation by distinct determinants in its C-terminal domain, Molecular Microbiology, 47, 171 (2003).
79. Paul, B. D., Kanhere, A., Chakraborty, A., Bansal, M., and Nagaraja, V., Identification of the domains for DNA binding and transactivation function of C protein from bacteriophage Mu, Proteins, 52, 272 (2003).
80. Ho, C. K., and Shuman, S., Bacteriophage T4 RNA ligase 2 (gp24.1) exemplifies a family of RNA ligases found in all phylogenetic domains.[erratum appears in Proc Natl Acad Sci USA 2002 October 15;99(21):13961], Proceedings of the National Academy of Sciences of the United States of America, 99, 12709 (2002).
81. Li, N., Sickmier, E. A., Zhang, R., Joachimiak, A., and White, S. W., The MotA transcription factor from bacteriophage T4 contains a novel DNA-binding domain: the ‘double wing’ motif, Molecular Microbiology, 43, 1079 (2002).
82. Pande, S., Makela, A., Dove, S. L., Nickels, B. E., Hochschild, A., and Hinton, D. M., The bacteriophage T4 transcription activator MotA interacts with the far-C-terminal region of the sigma70 subunit of Escherichia coli RNA polymerase, Journal of Bacteriology, 184, 3957 (2002).
83. Urbauer, J. L., Simeonov, M. F., Urbauer, R. J., Adelman, K., Gilmore, J. M., and Brody, E. N., Solution structure and stability of the anti-sigma factor AsiA: implications for novel functions, Proceedings of the National Academy of Sciences of the United States of America, 99, 1831 (2002).
84. Faber, C., Scharpf, M., Becker, T., Sticht, H., and Rosch, P., The structure of the coliphage HK022 Nun protein-lambda-phage boxB RNA complex. Implications for the mechanism of transcription termination, Journal of Biological Chemistry, 276, 32064 (2001).
85. Scharpf, M., Sticht, H., Schweimer, K., Boehm, M., Hoffmann, S., and Rosch, P., Antitermination in bacteriophage lambda. The structure of the N36 peptide-boxB RNA complex, European Journal of Biochemistry, 267, 2397 (2000).
86. Watnick, R. S., Herring, S. C., Palmer, A. G., 3rd, and Gottesman, M. E., The carboxyl terminus of phage HK022 Nun includes a novel zinc-binding motif and a tryptophan required for transcription termination, Genes & Development, 14, 731 (2000).
87. Bernal, R. A., Hafenstein, S., Olson, N. H., Bowman, V. D., Chipman, P. R., Baker, T. S., Fane, B. A., and Rossmann, M. G., Structural studies of bacteriophage alpha3 assembly, Journal of Molecular Biology, 325, 11 (2003).
88. Rentas, F. J., and Rao, V. B., Defining the bacteriophage T4 DNA packaging machine: evidence for a C-terminal DNA cleavage domain in the large terminase/packaging protein gp17, Journal of Molecular Biology, 334, 37 (2003).
89. Bleuit, J. S., Ma, Y., Munro, J., and Morrical, S. W., Mutations in a conserved motif inhibit single-stranded DNA binding and recombination mediator activities of bacteriophage T4 UvsY protein, Journal of Biological Chemistry, 279, 6077 (2004).
90. Melnyk, R. A., Kim, S., Curran, A. R., Engelman, D. M., Bowie, J. U., and Deber, C. M., The affinity of GXXXG motifs in transmembrane helix-helix interactions is modulated by long-range communication, Journal of Biological Chemistry, 279, 16591 (2004).
91. Papanikolopoulou, K., Forge, V., Goeltz, P., and Mitraki, A., Formation of highly stable chimeric trimers by fusion of an adenovirus fiber shaft fragment with the foldon domain of bacteriophage t4 fibritin, Journal of Biological Chemistry, 279, 8991 (2004).
92. Papanikolopoulou, K., Teixeira, S., Belrhali, H., Forsyth, V. T., Mitraki, A., and van Raaij, M. J., Adenovirus fibre shaft sequences fold into the native triple beta-spiral fold when N-terminally fused to the bacteriophage T4 fibritin foldon trimerisation motif, Journal of Molecular Biology, 342, 219 (2004).
93. Qu, Y., Hyman, P., Harrah, T., and Goldberg, E., In vivo bypass of chaperone by extended coiled-coil motif in T4 tail fiber, Journal of Bacteriology, 186, 8363 (2004).
94. van Raaij, M. J., Schoehn, G., Burda, M. R., and Miller, S., Crystal structure of a heat and protease-stable part of the bacteriophage T4 short tail fibre, Journal of Molecular Biology, 314, 1137 (2001).
95. Sam, M. D., Cascio, D., Johnson, R. C., and Clubb, R. T., Crystal structure of the excisionase-DNA complex from bacteriophage lambda, Journal of Molecular Biology, 338, 229 (2004).
96. Knowlton, J. R., Bubunenko, M., Andrykovitch, M., Guo, W., Routzahn, K. M., Waugh, D. S., Court, D. L., and Ji, X., A spring-loaded state of NusG in its functional cycle is suggested by X-ray crystallography and supported by site-directed mutants, Biochemistry, 42, 2275 (2003).
97. Sau, S., Chattoraj, P., Ganguly, T., Lee, C. Y., and Mandal, N. C., Cloning and sequencing analysis of the repressor gene of temperate mycobacteriophage L1, Journal of Biochemistry & Molecular Biology, 37, 254 (2004).
98. Shearwin, K. E., Dodd, I. B., and Egan, J. B., The helix-turn-helix motif of the coliphage 186 immunity repressor binds to two distinct recognition sequences, Journal of Biological Chemistry, 277, 3186 (2002).
99. Landthaler, M., and Shub, D. A., The nicking homing endonuclease I-BasI is encoded by a group I intron in the DNA polymerase gene of the Bacillus thuringiensis phage Bastille, Nucleic Acids Research, 31, 3071 (2003).
100. Lee, I., and Harshey, R. M., The conserved CA/TG motif at Mu termini: T specifies stable transpososome assembly, Journal of Molecular Biology, 330, 261 (2003).
101. Reiter, T. A., and Rusnak, F., Electrochemical studies of the mono-Fe, Fe—Zn, and Fe—Fe metalloisoforms of bacteriophage lambda protein phosphatase, Biochemistry, 43, 782 (2004).
102. Zhu, H., Yin, S., and Shuman, S., Characterization of polynucleotide kinase/phosphatase enzymes from Mycobacteriophages omega and Cjw1 and vibriophage KVP40, Journal of Biological Chemistry, 279, 26358 (2004).
103. Chan, A. Y., and Lim, B. L., Interaction of a putative transcriptional regulatory protein and the thermo-inducible cts-52 mutant repressor in the Bacillus subtilis phage phi 105 genome, Journal of Molecular Biology, 333, 21 (2003).
104. Goetzinger, K. R., and Rao, V. B., Defining the ATPase center of bacteriophage T4 DNA packaging machine: requirement for a catalytic glutamate residue in the large terminase protein gp17, Journal of Molecular Biology, 331, 139 (2003).
105. Lin, T. H., Tsai, K. C., and Lo, T. C., Homology modeling of the central catalytic domain of insertion sequence ISLC3 isolated from Lactobacillus casei ATCC 393, Protein Engineering, 16, 819 (2003).
106. Logan, D. T., Mulliez, E., Larsson, K. M., Bodevin, S., Atta, M., Garnaud, P.

E., Sjoberg, B. M., and Fontecave, M., A metal-binding site in the catalytic subunit of anaerobic ribonucleotide reductase, Proceedings of the National Academy of Sciences of the United States of America, 100, 3826 (2003).
107. Rodriguez, I., Lazaro, J. M., Salas, M., and de Vega, M., phi29 DNA polymerase residue Phe128 of the highly conserved (S/T)L×(2)h motif is required for a stable and functional interaction with the terminal protein, Journal of Molecular Biology, 325, 85 (2003).
108. Rogov, V. V., Lucke, C., Muresanu, L., Wienk, H., Kleinhaus, I., Werner, K., Lohr, F., Pristovsek, P., and Ruterjans, H., Solution structure and stability of the full-length excisionase from bacteriophage HK022, European Journal of Biochemistry, 270, 4846 (2003).
109. Wang, L. K., Ho, C. K., Pei, Y., and Shuman, S., Mutational analysis of bacteriophage T4 RNA ligase 1. Different functional groups are required for the nucleotidyl transfer and phosphodiester bond formation steps of the ligation reaction, Journal of Biological Chemistry, 278, 29454 (2003).
110. Yin, S., Ho, C. K., and Shuman, S., Structure-function analysis of T4 RNA ligase 2, Journal of Biological Chemistry, 278, 17601 (2003).
111. Romero, P., Lopez, R., and Garcia, E., Characterization of LytA-like N-acetylmuramoyl-L-alanine amidases from two new Streptococcus mitis bacteriophages provides insights into the properties of the major pneumococcal autolysin, Journal of Bacteriology, 186, 8229 (2004).
112. Calin-Jageman, I., and Nicholson, A. W., RNA structure-dependent uncoupling of substrate recognition and cleavage by Escherichia coli ribonuclease III, Nucleic Acids Research, 31, 2381 (2003).
113. Chan, E. Y., Goncalves, N. M., Haeusler, R. A., Hatch, A. J., Larson, J. W., Maletta, A. M., Yantz, G. R., Carstea, E. D., Fuchs, M., Wong, G. G., Gullans, S. R., and Gilmanshin, R., DNA mapping using microfluidic stretching and single-molecule detection of fluorescent site-specific tags, Genome Research, 14, 1137 (2004).
114. Dabrowska, K., Opolski, A., Wietrzyk, J., Switala-Jelen, K., Boratynski, J., Nasulewicz, A., Lipinska, L., Chybicka, A., Kujawa, M., Zabel, M., Dolinska-Krajewska, B., Piasecki, E., Weber-Dabrowska, B., Rybka, J., Salwa, J., Wojdat, E., Nowaczyk, M., and Gorski, A., Antitumor activity of bacteriophages in murine experimental cancer models caused possibly by inhibition of beta3 integrin signaling pathway, Acta Virologica, 48, 241 (2004).
115. Doulatov, S., Hodes, A., Dai, L., Mandhana, N., Liu, M., Deora, R., Simons, R. W., Zimmerly, S., and Miller, J. F., Tropism switching in Bordetella bacteriophage defines a family of diversity-generating retroelements.[see comment], Nature, 431, 476 (2004).
116. Labrie, S., Vukov, N., Loessner, M. J., and Moineau, S., Distribution and composition of the lysis cassette of Lactococcus lactis phages and functional analysis of bacteriophage u136 holin, FEMS Microbiology Letters, 233, 37 (2004).
117. Vukov, N., Moll, I., Blasi, U., Scherer, S., and Loessner, M. J., Functional regulation of the Listeria monocytogenes bacteriophage A118 holin by an intragenic inhibitor lacking the first transmembrane domain, Molecular Microbiology, 48, 173 (2003).
118. Vukov, N., Scherer, S., Hibbert, E., and Loessner, M. J., Functional analysis of heterologous holin proteins in a lambdaDeltaS genetic background, FEMS Microbiology Letters, 184, 179 (2000).
119. Barenboim, M., Chang, C. Y., dib Hajj, F., and Young, R., Characterization of the dual start motif of a class II holin gene, Molecular Microbiology, 32, 715 (1999).
120. Piersanti, S., Cherubini, G., Martina, Y., Salone, B., Avitabile, D., Grosso, F., Cundari, E., Di Zenzo, G., and Saggio, I., Mammalian cell transduction and internalization properties of lambda phages displaying the full-length adenoviral penton base or its central domain, Journal of Molecular Medicine, 82, 467 (2004).

The invention is claimed as follows:

1. A method for identifying a bacteriophage that lyses a bacterium comprising determining the presence of at least one copy of at least two oligonucleotides, or reverse complements thereof, of a motif set in the genome of said bacteriophage, wherein each of said at least two oligonucleotides is at least three nucleotides in length; hybridizes to either strand of the phage genome; is present in at least one copy in each phage of a phage test data set comprising at least three species-specific phage strains that lyse said bacterium; and is not present in a phage that does not lyse said bacterium.

2. The method of claim 1, wherein said bacterium is Listeria monocytogenes.

3. The method of claim 1, wherein said bacterium is a Salmonella.

4. The method of claim 1, wherein said motif set comprises at least three oligonucleotides.

5. The method of claim 1, wherein said motif set comprises at least four oligonucleotides.

6. The method of claim 1, wherein said motif set comprises at least five oligonucleotides.

7. The method of claim 1, wherein said motif set comprises at least six oligonucleotides.

8. The method of claim 1, wherein said motif set comprises at least seven oligonucleotides.

9. The method of claim 1, wherein said motif set comprises at least eight oligonucleotides.

10. The method of claim 1, wherein said motif set comprises at least nine oligonucleotides.

11. The method of claim 1, wherein said motif set comprises at least ten oligonucleotides.

12. The method of claim 1, wherein said phage test data set comprises at least four phage strains.

13. The method of claim 1, wherein said phage test data set comprises at least five phage strains.

14. The method of claim 1, wherein said phage test data set comprises at least six phage strains.

15. The method of claim 1, wherein said phage test data set comprises at least seven phage strains.

16. A bacteriophage comprising at least one copy of at least two oligonucleotides, or reverse complements thereof, of a motif set in the genome of said bacteriophage, wherein each of said at least two oligonucleotides is at least three nucleotides in length; hybridizes to either strand of the phage genome; is present in at least one copy in each phage of a phage test data set comprising at least three species-specific phage strains that lyse said bacterium; and is not present in a phage that does not lyse said bacterium identified by the method of any one of claims 1-15.

17. An oligonucleotide, or reverse complement thereof, contained within at least three species-specific bacteriophage that lyse a bacterium, of at least three bases in length.

18. The oligonucleotide of claim 17 which is at least four bases.

19. The oligonucleotide of claim 17 which is at least five bases.

20. The oligonucleotide of claim 17 which is at least six bases.

21. The oligonucleotide of claim 17 which is at least seven bases.

22. The oligonucleotide of claim 17 which is at least eight bases.

23. The oligonucleotide of claim 17 which is at least nine bases.

24. The oligonucleotide of claim 17 which is at least ten bases.

25. The oligonucleotide of claim 17 contained within at least four species-specific lytic phage.

26. The oligonucleotide of claim 17 contained within at least five species-specific lytic phage.

27. The oligonucleotide of claim 17 contained within at least six species-specific lytic phage.

28. The oligonucleotide of claim 17 contained within at least seven species-specific lytic phage.

29. A bacteriophage comprising at least one copy of at least two oligonucleotides of any one of claims 17-28, or reverse complements thereof, of a motif set in the genome of said bacteriophage, wherein each of said at least two oligonucleotides is at least three nucleotides in length; hybridizes to either strand of the phage genome; is present in at least one copy in each phage of a phage test data set comprising at least three species-specific phage strains that lyse said bacterium; and is not present in a phage that does not lyse said bacterium.

30. The bacteriophage of claim 29, wherein said motif set comprises at least three oligonucleotides.

31. The bacteriophage of claim 29, wherein said motif set comprises at least four oligonucleotides.

32. The bacteriophage of claim 29, wherein said motif set comprises at least five oligonucleotides.

33. The bacteriophage of claim 29, wherein said motif set comprises at least six oligonucleotides.

34. The bacteriophage of claim 29, wherein said motif set comprises at least seven oligonucleotides.

35. The oligonucleotide of claim 29, wherein said motif set comprises at least eight oligonucleotides.

36. The oligonucleotide of claim 29, wherein said motif set comprises at least nine oligonucleotides.

37. The oligonucleotide of claim 29, wherein said motif set comprises at least ten oligonucleotides.
